(19) United States
(12) Patent Application Publication
(10) Pub. No.: US 2005/0028227 A1
(43) Pub. Date: Feb. 3, 2005

(54) BSEP polypeptide variants and uses thereof

(75) Inventors: Ruitang Deng, Old Lyme, CT (US); Amy Y. Willis, Niantic, CT (US); Jinghai J. Xu, Westerly, RI (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/900,122

(22) Filed: Jul. 26, 2004

Related U.S. Application Data
(60) Provisional application No. 60/491,888, filed on Jul. 31, 2003.

(51) Int. Cl. C12Q 1/68; A01K 67/00; C07K 14/705

(52) U.S. Cl. 800/8; 435/6; 435/69.1; 435/320.1; 435/325; 530/350; 536/23.5

(57) ABSTRACT

The invention provides isolated nucleic acids encoding BSEP polypeptide variants, wherein the nucleic acids comprise a segment of nucleotides at a position which corresponds to the junction of Exon I and Exon II of a human BSEP polypeptide nucleotide sequence, and the polypeptide encoded by these nucleic acids. The invention also provides vectors, host cells, and transgenic animals comprising a nucleic acid encoding a BSEP polypeptide variant. The invention provides antibodies that specifically bind to the BSEP polypeptide variants of the invention. The invention also provides methods of identifying compounds that are substrates and/or modulators of the BSEP polypeptide variants of the invention. Finally, the invention provides methods of inhibiting the expression of a BSEP polypeptide variant of the invention.

### CROSS REFERENCE TO RELATED APPLICATIONS

This patent application claims priority of U.S. Provisional Patent Application No. 60/491,888 filed Jul. 31, 2003.

### FIELD OF THE INVENTION

This invention relates to the field of recombinant DNA technology and relates to nucleic acids encoding BSEP polypeptide variants, BSEP polypeptide variants and uses of these BSEP polypeptide variants and the nucleic acids encoding them.

### BACKGROUND OF THE INVENTION

Bile salt export pump (BSEP) is an ATP-dependent bile acid transporter that is responsible for the active transport of bile acid across the hepatocyte canalicular membrane into the bile duct. The gene encoding BSEP is formally known as ABCB11. BSEP was formerly known as “sister of p-glycoprotein.”

The human, rat, pig and mouse BSEP genes have been isolated. See, e.g., Strautnieks et al., Nature, Genetics 20:233-38 (1998), Gerloff et al., J. Biol. Chem., 273:10046-50 (1998); Green et al., Gene, 241:117-23 (2000); Childs et al., Cancer Res., 55:2029-34 (1995). The human BSEP gene encodes a 1321 amino acid polypeptide. It has been reported that the human BSEP gene is mutated in individuals having progressive familial intrahepatic cholestasis 2 (PFIC-2), a genetic disease causing extreme pruritus, growth failure and progression to cirrhosis in the first decade of life. Strautnieks et al., Nature Genetics, 20:233-38 (1998).

### SUMMARY OF THE INVENTION

This invention provides isolated nucleic acids encoding BSEP polypeptide variants. In one embodiment the invention provides isolated nucleic acids encoding BSEP polypeptide insert variants, wherein the nucleic acids comprise a segment of nucleotides at a position which corresponds to the junction between Exon I and Exon II of a human BSEP polypeptide nucleotide sequence.

This invention also provides isolated nucleic acids encoding a BSEP polypeptide insert variant, wherein the nucleic acid is at least 80% identical to a nucleic acid encoding a human BSEP polypeptide insert variant, wherein the nucleic acid encoding the human BSEP polypeptide insert variant comprises a segment of nucleotides at a position which corresponds to the junction between Exon I and Exon II of a human BSEP polypeptide nucleotide sequence.

The invention also provides vectors comprising nucleotides encoding a BSEP polypeptide variant. In one embodiment, the vectors comprise nucleotides encoding a BSEP polypeptide insert variant.

The invention also provides host cells comprising a nucleic acid encoding a BSEP polypeptide variant. In one embodiment the host cell comprises a nucleic acid encoding a BSEP polypeptide insert variant.

The invention also provides isolated BSEP polypeptide variants. In one embodiment the invention provides isolated BSEP polypeptide insert variants encoded by a nucleic acid comprising a segment of nucleotides at a position which corresponds to the junction between Exon I and Exon II of a BSEP polypeptide nucleotide sequence.

The invention also provides methods of preparing a BSEP polypeptide variant comprising culturing host cells described herein under conditions that permit expression of the BSEP polypeptide variant; and isolating the BSEP polypeptide variant, thereby preparing the BSEP polypeptide variant.

This invention provides antibodies, antibody chains, or fragments thereof, that specifically bind to a BSEP polypeptide variant, or a fragment of a BSEP polypeptide variant, and do not bind to a BSEP polypeptide.

The present invention also provides methods for detecting a BSEP polypeptide variant in a sample comprising: (a) contacting a suitable sample with a compound that specifically binds to the BSEP polypeptide variant; and (b) determining whether any compound is bound to the BSEP polypeptide variant, where the presence of any compound bound to the BSEP polypeptide variant detects the BSEP polypeptide variant in the sample.

The invention also provides methods for detecting a nucleic acid which encodes a BSEP polypeptide variant comprising: (a) contacting a suitable sample with a compound capable of specifically binding a nucleic acid encoding a BSEP polypeptide variant; and (b) determining whether any compound is bound to the nucleic acid, where the presence of compound bound to the nucleic acid detects the nucleic acid in the sample.

The present invention also provides for methods of determining whether a subject is expressing a BSEP polypeptide variant comprising: (a) contacting a suitable sample from the subject with a compound that specifically binds to the BSEP polypeptide variant; and (b) determining whether any compound is bound to the BSEP polypeptide variant, wherein the presence of the compound bound to the BSEP polypeptide variant indicates that the individual is expressing the BSEP polypeptide variant.

The invention also provides methods of detecting a nucleic acid which encodes a BSEP polypeptide variant comprising: (a) obtaining cDNA from mRNA obtained from a suitable sample; (b) amplifying the cDNA corresponding to the nucleic acid encoding the BSEP polypeptide or a portion of said nucleic acid; (c) comparing the amplified cDNA to the DNA of a nucleic acid known to encode a BSEP polypeptide, wherein a difference between the amplified cDNA and the DNA from a nucleic acid known to encode a BSEP polypeptide indicates the detection of a nucleic acid encoding a BSEP polypeptide variant in the sample.

In one embodiment a suitable sample is obtained from a subject, and the detection of the amplified cDNA indicates that the subject is expressing the BSEP polypeptide insert variant. The invention also provides methods of detecting a nucleic acid which encodes a BSEP polypeptide insert variant comprising: (a) obtaining cDNA from mRNA obtained from a suitable sample; (b) amplifying the cDNA corresponding to the insertion of the BSEP polypeptide insert variant or a portion of said insertion; and (c) comparing the amplified cDNA to the DNA of a nucleic acid known to encode a BSEP polypeptide, wherein the presence of an insertion in the amplified cDNA indicates the detection of a nucleic acid encoding the BSEP polypeptide insert variant.

The present invention provides methods of identifying compounds that are substrates and/or modulators of the BSEP polypeptide variants of the invention.

The invention provides methods for determining whether a compound is a substrate or modulator of a BSEP polypeptide variant comprising: (a) contacting the compound with a BSEP polypeptide variant; and (b) determining whether any compound binds to the BSEP polypeptide variant, wherein the presence of compound bound to the BSEP polypeptide variant indicates that the compound is a substrate or modulator of the BSEP polypeptide variant.

The invention also provides methods for determining whether a compound is a substrate or modulator of a BSEP polypeptide variant comprising: (a) contacting the BSEP polypeptide variant with a known binder and the compound; (b) measuring the amount of binder bound to the BSEP polypeptide variant; and (c) comparing the amount of binder bound to the BSEP polypeptide variant in step (b) with the amount of binder bound to the BSEP polypeptide variant in the absence of the compound, and determining whether the presence of the compound increased or decreased the amount of binder bound to the BSEP polypeptide variant, wherein an increase or a decrease in the amount of binder bound to the BSEP polypeptide variants indicates that the compound is a substrate or modulator of BSEP.

The invention also provides methods for determining whether a compound is a substrate of a BSEP polypeptide variant comprising: (a) contacting the compound with the BSEP polypeptide variant and ATP; and (b) detecting the ATPase activity of the BSEP polypeptide insert variant, wherein the detection of ATPase activity indicates that the compound is a substrate of the BSEP polypeptide variant.

The invention also provides methods for determining whether a compound is an modulator of a BSEP polypeptide variant comprising: (a) contacting the BSEP polypeptide variant with a substrate of the BSEP polypeptide variant, a compound and ATP; (b) measuring the ATPase activity of the BSEP polypeptide variant; and (c) comparing the ATPase activity of the BSEP polypeptide variant in step (b) with the ATPase activity of the BSEP polypeptide variant in the absence of the compound, and determining whether the compound modulated the ATPase activity of the BSEP polypeptide variant, wherein an increase or a decrease in the ATPase activity indicates that the compound is a substrate or modulator of the BSEP polypeptide variant.

The present invention also provides methods for determining whether a compound is a substrate of BSEP comprising: (a) contacting the compound with the BSEP polypeptide variant; (b) determining whether the compound is transported by the BSEP polypeptide variant, wherein the transport of the compound by the BSEP polypeptide variant indicates that the compound is a substrate of the BSEP polypeptide variant.

The invention also provides methods for determining whether a compound is a substrate or modulator of a human BSEP polypeptide variant comprising: (a) contacting the compound with the BSEP polypeptide variant and a known substrate of the BSEP polypeptide variant; (b) measuring the activity of the BSEP polypeptide variant with respect to the known substrate; and (c) comparing the activity of the BSEP polypeptide variant with respect to the known substrate in step (b) with the activity of the BSEP polypeptide with respect to the known substrate in the absence of the compound, wherein an increase or a decrease in transport of activity of the BSEP polypeptide insert variant with respect to the known substrate indicates that the compound is a substrate or modulator of the BSEP polypeptide insert variant.

The invention also provides for methods of inhibiting the expression of a BSEP polypeptide variant in a cell comprising contacting the cell with a compound capable of inhibiting the expression of the BSEP polypeptide variant. In one embodiment, the compound capable of inhibiting the expression of a BSEP polypeptide variant is an antisense oligonucleotide capable of specifically hybridizing to the BSEP polypeptide variant. In another embodiment, the compound capable of inhibiting the expression of a BSEP polypeptide variant is an RNAi construct.

The invention further provides a non-human transgenic animal whose somatic and germ cells contain a heterologous nucleic acid encoding a BSEP polypeptide variant operably-linked to a promoter, said nucleic acid comprising a segment of nucleotide at a position which corresponds to the junction between Exon I and Exon II of a human BSEP polypeptide nucleotide sequence.

### DETAILED DESCRIPTION OF THE INVENTION

The articles “a,” “an” and “the” are used herein to refer to one or to more than one (i.e., to at least one) of the grammatical object of the article.

Nucleic Acids

This invention provides isolated nucleic acids encoding BSEP polypeptide variants.

In one embodiment, the BSEP polypeptide variants will be human BSEP polypeptide variants. In another embodiment, the BSEP polypeptide variants will be rat BSEP polypeptide variants. In another embodiment, the BSEP polypeptide variants will be mouse BSEP polypeptide variants. In another embodiment, the BSEP polypeptide variants may be derived from other species including, but not limited to, dogs, guinea pigs, ferrets and rabbits.

In one embodiment the BSEP polypeptide variants will be insert variants.

In one embodiment the invention provides isolated nucleic acids encoding BSEP polypeptide insert variants, wherein the nucleic acids comprise a segment of nucleotides at a position which corresponds to the junction between Exon I and Exon II of a human BSEP polypeptide nucleotide sequence.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the coexisting materials in the natural system, is isolated. Such polynucleotides could be part of a vector and/or such polynucleotides or polypeptides could be part of a composition, and still be isolated in that such vector or composition is not part of the environment in which it is found in nature.

The term “nucleic acid,” as used herein, refers to either DNA or RNA. The nucleic acid can be isolated from natural sources or can be a product of chemical synthetic procedures. The term “nucleic acid” should also be understood to include, as equivalents, analogs of either RNA or DNA made from nucleotide analogs. The nucleic acid can be single-stranded or double-stranded, and if single-stranded may be the coding strand or non-coding (antisense) strand.

“Nucleic acid,” “nucleic acid sequence,” “nucleotide sequence,” or “polynucleotide sequence” refers to a single- or double-stranded polymer of deoxyribonucleotide or ribonucleotide bases read from the 5′ to the 3′ end.

The phrase “nucleic acid encoding,” as used herein, refers to a nucleic acid, which directs the expression of a specific protein or peptide. The nucleic acid sequences include both the DNA strand sequence that is transcribed into RNA, and the RNA sequence that is translated into protein.

As used herein, the term “polypeptide” refers to two or more amino acids linked by a peptide bond between the α-carboxyl group of one amino acid and the α-amino group of the next amino acid. The polypeptide can be isolated from natural sources, can be a produced by recombinant techniques from a eukaryotic or prokaryotic host, or can be a product of synthetic procedures.

The term “BSEP polypeptide,” as used herein, refers to any BSEP polypeptide previously known. Examples of amino acid sequences of human BSEP polypeptides can be found in GenBank® (see, e.g., Accession Nos. AAC77455, NP_{—}003733, AAD28285). All of these human BSEP polypeptides comprise 1321 amino acids. Examples of nucleic acid sequences encoding human BSEP polypeptides can be found in GenBank® (see, e.g., Accession Nos. AF091582, NM_{—}003742 and AF136523). The junction between Exon I and Exon II in the nucleotide sequence disclosed in GenBank® Accession No. AF091582 is between nucleotides 202 and 203. The junction between Exon I and Exon II in the nucleotide sequence disclosed in GenBank® Accession No. NM_{—}003742 is between nucleotides 202 and 203. The junction between Exon I and Exon II in the nucleotide sequence disclosed in GenBank® Accession No. AF136523 is between nucleotides 203 and 204.

As used herein, the phrase “BSEP polypeptide nucleotide sequence” refers to a nucleic acid encoding a BSEP polypeptide.

In one embodiment, the human BSEP polypeptide is encoded by the nucleotide sequence disclosed in SEQ ID NO:1. In SEQ ID NO:2, the junction between Exon I and Exon II, corresponds to the position between nucleotides 76 and 77.

In one embodiment, the human BSEP polypeptide comprises the amino acid sequence disclosed in SEQ ID NO:2, or an amino acid sequence that substantially corresponds to this sequence.

In one embodiment, the human BSEP polypeptide is encoded by the nucleotide sequence disclosed in SEQ ID NO:1, or a nucleotide sequence that substantially corresponds to this sequence.

A sequence that “substantially corresponds” to another sequence is a sequence that allows single amino acid or nucleotide substitutions, deletions and/or insertions. In one embodiment, sequences that substantially correspond have 80% sequence identity. In another embodiment, sequences that substantially correspond have 85% sequence identity. In another embodiment, sequences that substantially correspond have 90% sequence identity. In another embodiment, sequences that substantially correspond have 95% sequence identity. In another embodiment, sequences that substantially correspond have 97% sequence identity. In another embodiment, sequences that substantially correspond have 99% sequence identity.

As used herein, the term “% sequence identity” refers to the sequence relationship between two or more nucleic acid molecules or polypeptides. The terms “identical” or percent “identity”, in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. This definition also refers to the complement of a test sequence, provided that the test sequence has a designated or substantial identity to a reference sequence.

Comparisons between the sequences of two or more polynucleotides or polypeptides can be performed using the local homology algorithm of Smith and Waterman, Adv. Appl. Math. 2: 482 (1981), by the homology alignment algorithm of Needleman and Wunsch, J. Mol. Biol. 48: 443 (1970), or the method of Pearson and Lipman, PNAS 85: 2444 (1988). Computer programs implementing these methods can be used and include, BLAST, GAP, BESTFIT, FASTA, and TFASTA which are offered in the Wisconsin Genetics Software Package, Genetics Computer Group, 575 Science Dr., Madison, Wis.

The term “BSEP polypeptide variant,” as used herein, refers to a BSEP polypeptide comprising amino acid insertions, deletions and/or substitutions with respect to the BSEP polypeptides previously known. Examples of previously known BSEP polypeptides can be found in GenBank® Accession Nos. AAC77455, NP_{—}003733 and AAD28285.

The term “BSEP polypeptide insert variant,” as used herein, refers to a human BSEP polypeptide comprising an insertion of nucleotides at a position which corresponds to the junction between Exon I and Exon II of a human BSEP polypeptide nucleotide sequence. In one embodiment, the human BSEP polypeptide insert variant of the invention comprises more than 1321 amino acids.

In one embodiment of the nucleic acids described herein, the segment of nucleotides at the position that corresponds to the junction between Exon I and Exon II comprises nucleotides encoding the amino acid sequence set forth in SEQ ID NO:11, or such amino acid sequence comprising at least one conservative amino acid substitution. In one embodiment, the segment of nucleotides encoding the amino acid sequence set forth in SEQ ID NO:1 comprises the nucleotide sequence set forth in SEQ ID NO:12.

In one embodiment, the nucleic acid encodes a human BSEP polypeptide insert variant comprising the amino acid sequence set forth in SEQ ID NO:4. In another embodiment, the nucleic acid encoding SEQ ID NO:4 comprises the nucleotide sequence set forth in SEQ ID NO:3.

In another embodiment, the nucleic acid encodes a human BSEP polypeptide insert variant comprising the amino acid sequence set forth in SEQ ID NO:4 with a conservative amino acid substitution. In another embodiment, the nucleic acid encodes a human BSEP polypeptide insert variant comprising 1, 2, 3, 4, 5, 6, 7, 8, 9 or 10 conservative amino acid substitutions with respect to SEQ ID NO:4. In another embodiment, the nucleic acid encodes a human BSEP polypeptide insert variant comprising 11, 12, 13, 14, 15, 16, 17, 18, 19, or 20 conservative amino acid substitutions with respect to SEQ ID NO:4. In another embodiment, the nucleic acid encodes a human BSEP polypeptide insert variant comprising 21, 22, 23, 24, 25, 26, 27, 28, 29, or 30 conservative amino acid substitutions with respect to SEQ ID NO:4. In yet another embodiment, the nucleic acid encodes a human BSEP polypeptide comprising more than 30 conservative amino acid substitutions with respect to SEQ ID NO:4.

In another embodiment, the nucleic acid encodes a human BSEP polypeptide insert variant comprising the amino acid sequence set forth in SEQ ID NO:8. In another embodiment, the nucleic acid encoding SEQ ID NO:8 comprises the nucleotide sequence set forth in SEQ ID NO:7.

In another embodiment, the nucleic acid encodes a human BSEP polypeptide insert variant comprising the amino acid sequence set forth in SEQ ID NO:8 with a conservative amino acid substitution. In another embodiment, the nucleic acid encodes a human BSEP polypeptide insert variant comprising 1, 2, 3, 4, 5, 6, 7, 8, 9 or 10 conservative amino acid substitutions with respect to SEQ ID NO:8. In another embodiment, the nucleic acid encodes a human BSEP polypeptide insert variant comprising 11, 12, 13, 14, 15, 16, 17, 18, 19, or 20 conservative amino acid substitutions with respect to SEQ ID NO:8. In another embodiment, the nucleic acid encodes a human BSEP polypeptide insert variant comprising 21, 22, 23, 24, 25, 26, 27, 28, 29, or 30 conservative amino acid substitutions with respect to SEQ ID NO:8. In yet another embodiment, the nucleic acid encodes a human BSEP polypeptide comprising more than 30 conservative amino acid substitutions with respect to SEQ ID NO:8.

The phrase “conservative amino acid substitution” refers to the substitution of one amino acid with a different amino acid having certain common properties. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include: (i) a charged group, consisting of Glu and Asp, Lys, Arg and His; (ii) a positively-charged group, consisting of Lys, Arg and His; (iii) a negatively-charged group, consisting of Glu and Asp; (iv) an aromatic group, consisting of Phe, Tyr and Trp; (v) a nitrogen ring group, consisting of His and Trp; (vi) a large aliphatic nonpolar group, consisting of Val, Leu and Ile; (vii) a slightly-polar group, consisting of Met and Cys; (viii) a small-residue group, consisting of Ser, Thr, Asp, Asn, Gly, Ala, Glu, Gln and Pro; (ix) an aliphatic group consisting of Val, Leu, Ile, Met and Cys; and (x) a small hydroxyl group consisting of Ser and Thr.

In another embodiment, the nucleic acids described herein encode BSEP polypeptide insert variants comprising an amino acid sequence substantially corresponding to the amino acid sequence set forth in SEQ ID NO:4 or SEQ ID NO:8.

The nucleic acids of the invention encoding BSEP polypeptide variants of the invention are not limited to the nucleic acids disclosed herein. Now that the existence of human BSEP polypeptide variants, has been disclosed by the present document, additional nucleic acid sequences encoding other BSEP polypeptide variants can be discovered using methods known to a person having ordinary skill in the art. For example, additional BSEP polypeptide variants can be discovered by analysis of PCR (polymerase chain reaction) products from segments of total RNA and mRNA (see, e.g., discussion infra, regarding methods of detecting nucleic acids encoding BSEP polypeptide variants). Additional BSEP polypeptide variants can be also discovered by directly cloning, and sequencing, nucleic acids encoding BSEP polypeptides from a cDNA library. These methods could be used to identify BSEP polypeptide variants in other species.

The invention also provides isolated nucleic acids capable of hybridizing under high stringency conditions to a nucleic acid described herein.

The invention provides an isolated nucleic acid capable of hybridizing under high stringency conditions to a nucleic acid encoding a human BSEP polypeptide insert variant, wherein the nucleic acid comprises a segment of nucleotides at a position which corresponds to the junction between Exon I and Exon II of a human BSEP polypeptide nucleotide sequence.

This invention also provides an isolated nucleic acid capable of hybridizing under high stringency conditions to a nucleic acid encoding SEQ ID NO:6, or a fragment thereof.

This invention also provides an isolated nucleic acid capable of hybridizing under high stringency conditions to SEQ ID NO:5, or a fragment thereof.

This invention also provides an isolated nucleic acid capable of hybridizing under high stringency conditions to a nucleic acid encoding SEQ ID NO:10, or a fragment thereof.

This invention also provides an isolated nucleic acid capable of hybridizing under high stringency conditions to SEQ ID NO:9, or a fragment thereof.

This invention also provides an isolated nucleic acid capable of hybridizing under high stringency conditions to a nucleic acid encoding SEQ ID NO:11, or a fragment thereof.

This invention also provides an isolated nucleic acid capable of hybridizing under high stringency conditions to SEQ ID NO:12, or a fragment thereof.

As used here, “hybridization,” means hydrogen bonding between complementary nucleoside or nucleotide bases. “Complementary,” as used herein, refers to the capacity for precise pairing between two nucleosides. The oligonucletoide and the DNA or RNA are complementary to each other when a sufficient number of corresponding positions in each molecule are occupied by nucleotides which can hydrogen bond with each other. Thus, “specifically hybridizing to” and “complementary” are terms which are used to indicate a sufficient degree of complementarity or precise pairing such that stable and specific binding occurs between the oligonucleotide and the DNA or RNA target.

Hybridization can be determined by using, e.g., standard nucleic acid hybridization techniques. Stringency can be controlled, e.g., by varying salt and/or denaturant concentrations and/or by varying the temperature. See, for example, Molecular Cloning, a Laboratory Manual, ed. by Sambrook, Fritsch and Maniatis, Cold Spring Harbor Laboratory Press, 1989.

High stringency hybridization conditions are selected at about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, high stringency conditions will be those in which the salt concentration is at least about 0.02 molar at pH 7 and the temperature is at least about 60° C. As other factors may significantly affect the stringency of hybridization, including, among others, base composition and size of the complementary strands, the presence of organic solvents, i.e. salt or formamide concentration, and the extent of base mismatching. When used in the claims, the term “high stringency conditions” refers to conditions involving hybridization at about 68° C. in a 6×SSC solution, followed by washing at about 68° C. in a 0.6×SSC solution or similar conditions (1×SSC is 0.15 M sodium chloride and 0.015 M sodium citrate). Those skilled in the art will recognize, based upon the present description, how such conditions can be varied to alter specificity and selectivity to achieve higher or lower stringency conditions.

This invention also provides isolated nucleic acids encoding a BSEP polypeptide insert variant, wherein the nucleic acid is at least 80% identical to a nucleic acid encoding a human BSEP polypeptide insert variant, wherein the nucleic acid encoding the human BSEP polypeptide insert variant comprises a segment of nucleotides at a position which corresponds to the junction between Exon I and Exon II of a human BSEP polypeptide nucleotide sequence. In increasingly more preferred embodiments, rather than 80%, the percent identity is 85%, 90%, 95%, 97%, or 99%.

In one embodiment, the invention provides isolated nucleic acids encoding a BSEP polypeptide insert variant, wherein the nucleic acid is at least 80% identical to a nucleic acid encoding a human BSEP polypeptide comprising the amino acid sequence set forth in SEQ ID NO:4, wherein the nucleic acid encoding the human BSEP polypeptide insert variant comprises a segment of nucleotides at a position which corresponds to the junction between Exon I and Exon II of a human BSEP polypeptide nucleotide sequence. In increasingly more preferred embodiments, rather than 80%, the percent identity is 85%, 90%, 95%, 97%, or 99%.

In one embodiment, the invention provides isolated nucleic acids encoding a BSEP polypeptide insert variant, wherein the nucleic acid is at least 80% identical to a nucleic acid encoding a human BSEP polypeptide comprising the amino acid sequence set forth in SEQ ID NO:8, wherein the nucleic acid encoding the human BSEP polypeptide insert variant comprises a segment of nucleotides at a position which corresponds to the junction between Exon I and Exon II of a human BSEP polypeptide nucleotide sequence. In increasingly more preferred embodiments, rather than 80%, the percent identity is 85%, 90%, 95%, 97%, or 99%.

The nucleic acids described herein can be labeled with a detectable marker. Detectable markers include, but are not limited to: a radioactive marker, a colorimetric marker, a luminescent marker, an enzyme marker and a fluorescent marker. Radioactive markers include, but are not limited to, ³H, ¹⁴C, ³²P, ³³P, ³⁵S, ³⁶Cl, ⁵¹Cr, ⁵⁷Co, ⁵⁹Co, ⁵⁹Fe, ⁹⁰Y, ¹²⁵I, ¹³¹I, and ¹⁸⁶Re. Fluorescent markers include, but are not limited to, fluorescein, rhodamine and auramine. Colorimetric markers include, but are not limited to, biotin and digoxigenin. Any suitable method for attaching markers to nucleic acids may be used with the nucleotides of the invention, and many such methods are well known in the art.

Further, the invention provides nucleic acids complementary to the nucleic acids disclosed herein. By a nucleic acid sequence “homologous to” or “complementary to”, it is meant a nucleic acid that selectively hybridizes, duplexes or binds to a target nucleic acid sequence. For example, adenine is complementary to thymine as they can form two hydrogen bonds. Similarly, guanine and cytosine are complementary since they can form three hydrogen bonds. A nucleic acid sequence, which is homologous to a target sequence, can include sequences, which are shorter or longer than the target sequence as long as they meet the functional test set forth.

It will be readily understood by those skilled in the art and it is intended here, that when reference is made to particular sequence listings, such reference includes sequences which substantially correspond to its complementary sequence and those described including allowances for minor sequencing errors, single base changes, deletions, substitutions and the like, such that any such sequence variation corresponds to the nucleic acid encoding the polypeptide to which the relevant sequence listing relates.

Vectors

The invention also provides vectors comprising nucleotides encoding a BSEP polypeptide variant. In one embodiment, the vectors comprise nucleotides encoding a human BSEP polypeptide variant. In one embodiment, the vectors comprise nucleotides encoding a BSEP polypeptide insert variant. In one embodiment, the vectors comprise the nucleotide sequences described herein. The vectors include, but are not limited to, a virus, plasmid, cosmid, λ phage or a yeast artificial chromosome (YAC).

In accordance with the invention, numerous vector systems may be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance, (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Exemplary vectors include, for example, those described by Okayama and Berg, Mol. Cell. Biol. 3:280 (1983).

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity. Expression of the gene encoding a BSEP polypeptide variant results in production of the BSEP polypeptide variant.

Methods and conditions for culturing the resulting transfected cells and for recovering the BSEP polypeptide variant so produced are well known to those skilled in the art, and may be varied or optimized depending upon the specific expression vector and mammalian host cell employed, based upon the present description.

Cells

The invention also provides host cells comprising a nucleic acid encoding a BSEP polypeptide variant. In one embodiment, the BSEP polypeptide variant is a human BSEP polypeptide variant. In one embodiment the BSEP polypeptide variant is an insert variant.

In one embodiment, the host cells are genetically engineered to comprise nucleic acids encoding BSEP polypeptide variants.

In another embodiment, the nucleic acid encoding the BSEP polypeptide variant can be situated on an episome in a host cell. An “episome” is any of a group of genetic elements consisting of DNA and capable of giving selective advantage to the bacteria in which they occur. Episomes may be attached to the bacterial cell membrane or become part of the chromosome. Cells with episomes act like males during conjugation, a mating process in certain bacteria. During conjugation, cells lacking the episome may receive either the episome or the episome plus the genes to which it is attached. Experiments involving gene transfers from cells in which episomes have been incorporated in the chromosomes have been used to determine the locations of genes on the chromosome.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

Host cells comprising any of the nucleic acids described herein are useful, e.g., for identifying substrates and modulators of the BSEP polypeptide variants, and/or for identifying potentially toxic compounds or drugs.

BSEP Polypeptides Variants

The invention also provides isolated BSEP polypeptide variants. As discussed above, the term “BSEP polypeptide variant,” as used herein, refers to a BSEP polypeptide comprising amino acid insertions, deletions and/or substitutions with respect to the BSEP polypeptides previously known. In one embodiment, the invention provides isolated human BSEP polypeptide insert variants.

In one embodiment, the invention provides isolated BSEP polypeptide variants. In one embodiment, the invention provides isolated rat BSEP polypeptide insert variants. In one embodiment, the invention provides isolated mouse BSEP polypeptide insert variants. In other embodiments the isolated BSEP polypeptide variants will be derived from other species, including but not limited to, dogs, pigs, guinea pigs and rabbits.

In one embodiment the invention provides isolated BSEP polypeptide insert variants encoded by a nucleic acid comprising a segment of nucleotides at a position which corresponds to the junction between Exon I and Exon II of a nucleic acid encoding a BSEP polypeptide.

In one embodiment the invention provides isolated human BSEP polypeptide insert variants encoded by a nucleic acid comprising a segment of nucleotides at a position which corresponds to the junction between Exon I and Exon II of a human BSEP polypeptide nucleotide sequence. In one embodiment, the segment of nucleotides encodes the amino acid sequence set forth in SEQ ID NO:11. In another embodiment, the segment of nucleotides encodes an amino acid sequence having one or more conservative amino acid substitutions with respect to the amino acid sequence set forth in SEQ ID NO:11.

In one embodiment, the isolated BSEP polypeptide insert variant comprises the amino acid sequence set forth in SEQ ID NO:4. In one embodiment, the isolated human BSEP polypeptide comprises the amino acid sequence set forth in SEQ ID NO:8.

In another embodiment, the isolated BSEP polypeptide insert variant comprises an amino acid sequence having one or more conservative amino acid substitutions with respect to the sequence disclosed in SEQ ID NO:4. In one embodiment, the isolated BSEP polypeptide variant comprises 1, 2, 3, 4, 5, 6, 7, 8, 9 or 10 conservative amino acid substitutions relative to SEQ ID NO:4. In another embodiment the isolated BSEP polypeptide variant comprises 11, 12, 13, 14, 15, 16, 17, 18, 19 or 20 conservative amino acid substitutions relative to SEQ ID NO:4. In another embodiment the isolated BSEP polypeptide variant comprises 20, 21, 22, 23, 24, 25, 26, 27, 28, 29, or 30 conservative amino acid substitutions relative to SEQ ID NO:4. In yet another embodiment, the isolated BSEP polypeptide variant comprises more than 30 conservative amino acid substitutions relative to SEQ ID NO:4.

In another embodiment, the isolated BSEP polypeptide insert variant comprises an amino acid sequence having one or more conservative amino acid substitutions with respect to the sequence disclosed in SEQ ID NO:8. In one embodiment the isolated BSEP polypeptide variant comprises 1, 2, 3, 4, 5, 6, 7, 8, 9 or 10 conservative amino acid substitutions relative to SEQ ID NO:8. In another embodiment the isolated BSEP polypeptide variant comprises 11, 12, 13, 14, 15, 16, 17, 18, 19 or 20 conservative amino acid substitutions relative to SEQ ID NO:8. In another embodiment the isolated BSEP polypeptide variant comprises 20, 21, 22, 23, 24, 25, 26, 27, 28, 29, or 30 conservative amino acid substitutions relative to SEQ ID NO:8. In yet another embodiment, the isolated BSEP polypeptide variant comprises more than 30 conservative amino acid substitutions relative to SEQ ID NO:1.

In another embodiment, the isolated BSEP polypeptide insert variant comprises C an amino acid sequence substantially corresponding to the amino acid sequence set forth in SEQ ID NO:4 or SEQ ID NO:8.

The BSEP polypeptide variants can be isolated from natural sources, or can be a product of chemical synthetic procedures, or can be produced by recombinant techniques from a prokaryotic or eukaryotic host.

The BSEP polypeptide variants can be used, for example, during a drug screening and development process, e.g., to assess the potential toxic impact of a compound.

Methods of Preparing BSEP Polypeptide Variants

The invention also provides methods of preparing BSEP polypeptide variants comprising culturing host cells under conditions that permit expression of the BSEP polypeptide variant; and isolating the BSEP polypeptide variant, thereby preparing the BSEP polypeptide variant. In one embodiment, the invention provides a method of preparing a human BSEP polypeptide variant. In one embodiment, the invention provides a method of preparing a BSEP polypeptide insert variant. Procedures for preparing a polypeptide using the above describe method are well known to those skilled in the art. See, for example, Membrane Protein Purification and Crystallization, ed. by Hunte, Jagow and Schagger, Academic Press, 2002.

Antibodies

This invention provides antibodies, antibody chains, or fragments thereof that specifically bind to a BSEP polypeptide variant or a fragment of a BSEP polypeptide variant. In one embodiment the BSEP polypeptide variant to which the antibody binds is a human BSEP polypeptide variant. In one embodiment the BSEP polypeptide variant to which the antibody binds is BSEP polypeptide insert variant.

The term “specifically binds,” as use herein, refers to a binding reaction that is determinative of the presence of a target (such a specific polypeptide or nucleic acid) in a population of proteins and other biologics.

In one embodiment, the antibody specifically binds a BSEP polypeptide comprising the amino acid sequence set forth in SEQ ID NO:11. In one embodiment, the invention provides an antibody that specifically binds to a human BSEP polypeptide insert comprising the amino acid sequence set forth in SEQ ID NO:4. In another embodiment, the invention provides an antibody that specifically binds to a human BSEP polypeptide insert variant comprising the amino acid sequence set forth in SEQ ID NO:8.

In the context of the present invention, antibodies specifically binding to the BSEP polypeptide insert variants can be screened and tested to identify those antibodies that specifically recognize human BSEP polypeptide insert variants, but do not recognize the previously known human BSEP polypeptides. The antibodies can be screened using methods known to people skilled in the art such as, for example, Western blot, immunoprecipitation and ELISA (enzyme-linked immunosorbent assay).

In one embodiment, the invention provides an antibody that specifically binds to the human BSEP polypeptide insert variant of SEQ ID NO:4, but does not bind to the human BSEP polypeptide of SEQ ID NO:2. In another embodiment, the invention provides for an antibody that specifically binds to the human BSEP polypeptide insert variant of SEQ ID NO:4, but does not bind to the human BSEP polypeptide of SEQ ID NO:2 and does not bind to the human BSEP polypeptide insert variant of SEQ ID NO:8.

In one embodiment, the invention provides an antibody that specifically binds to the human BSEP polypeptide insert variant of SEQ ID NO:8, but does not bind to the human BSEP polypeptide of SEQ ID NO:2. In another embodiment, the invention provides an antibody that specifically binds to the human BSEP polypeptide insert variant of SEQ ID NO:8, but does not bind to the human BSEP polypeptide of SEQ ID NO:2 and does not bind to the human BSEP polypeptide insert variant of SEQ ID NO:4.

The antibodies may be polyclonal or monoclonal. Ponoclonal or molyclonal antibodies can be made using standard protocols (see, for example, Antibodies: A Laboratory Manual, ed. by Harlow and Lane (Cold Spring Harbor Press: 1988)). A subject such as a mammal, including but not limited to a mouse, a hamster, a rat, a goat, a rabbit or a human can be immunized with an immunogenic form of the human BSEP polypeptide variant. In one embodiment, the subject will be injected with a human BSEP polypeptide insert variant comprising the amino acid sequence set forth in SEQ ID NO:4, or a fragment thereof. In another embodiment, the subject will be injected with a human BSEP polypeptide insert variant comprising the amino acid sequence set forth in SEQ ID NO:8, or a fragment thereof. Techniques for conferring immunogenicity on a protein or peptide include conjugation to carriers or other techniques well known in the art. See, for example, Antibodies, a Laboratory Manual, ed. by Harlow and Lane, Cold Spring Harbor Press, 1988.

Following immunization of a subject with an antigenic preparation of a polypeptide, antisera can be obtained and, if desired, polyclonal antibodies isolated from the serum. To produce monoclonal antibodies, antibody-producing cells (lymphocytes) can be harvested from an immunized subject and fused by standard somatic cell fusion procedures with immortalizing cells such as myeloma cells to yield hybridoma cells. Such techniques are well known in the art, and include, for example, the hybridoma technique (originally developed by Kohler and Milstein, Nature, 256: 495-97 (1975)), the human B cell hybridoma technique (Kozbar et al., Immunology Today, 4: 72 (1983)), and the EBV-hybridoma technique to produce human monoclonal antibodies (Cole et al., Monoclonal Antibodies and Cancer Therapy, Alan R. Liss, Inc. pp. 77-96 (1985)). Hybridoma cells can be screened immunochemically for production of antibodies specifically reactive with a particular polypeptide and monoclonal antibodies isolated from a culture comprising such hybridoma cells.

The antibodies may be of the IgM, IgA, IgE or IgG class or subclasses thereof. The above antibody fragment includes but is not limited to Fab, Fab′, (Fab′) 2, Fv and single chain antibodies.

This invention provides an isolated antibody light chain of the above antibody, or fragment or oligomer thereof. This invention also provides an isolated antibody heavy chain of the above antibody, or fragment or oligomer thereof. This invention also provides one or more CDR regions of the above antibody. In one embodiment, the antibody is derivatized. In another embodiment, the antibody is a human antibody. In one embodiment, antibody is humanized.

As used herein, “oligomer” means a complex of 2 or more subunits.

As used herein, “CDR” or “complementarity determining region” means a highly variable sequence of amino acids in the variable domain of an antibody.

As used herein, a “derivatized” antibody is one that has been modified. Methods of derivatization include but are not limited to the addition of a fluorescent moiety, a radionucleotide, a toxin, an enzyme or an affinity ligand such as biotin.

As used herein, “humanized,” describes antibodies wherein some, most or all of the amino acids outside the CDR regions are replaced with corresponding amino acids derived from human immunoglobulin molecules. In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgGI, IgG2, IgG3, IgG4, IgA, IgE and IgM molecules. A “humanized” antibody would retain a similar antigenic specificity as the original antibody. One skilled in the art would know how to make the humanized antibodies of the subject invention. Various publications, several of which are hereby incorporated by reference into this application, also describe how to make humanized antibodies. For example, the methods described in U.S. Pat. No. 4,816,567 comprise the production of chimeric antibodies having a variable region of one antibody and a constant region of another antibody. See also Berger et al., American J. of the Medical Sciences 324(1):14-30, 2002.

U.S. Pat. No. 5,225,539 describes another approach for the production of a humanized antibody. This patent describes the use of recombinant DNA technology to produce a humanized antibody wherein the CDRs of a variable region of one immunoglobulin are replaced with the CDRs from an immunoglobulin with a different specificity such that the humanized antibody would recognize the desired target but would not be recognized in a significant way by the human subject's immune system. Specifically, site directed mutagenesis is used to graft the CDRs onto the framework.

Other approaches for humanizing an antibody are described in U.S. Pat. Nos. 5,585,089 and 5,693,761, as well as in International Patent Publication No. WO 90/07861, all of which describe methods for producing humanized immunoglobulins. These have one or more CDRs and possible additional amino acids from a donor immunoglobulin and a framework region from an accepting human immunoglobulin. These documents describe methods to increase the affinity of an antibody for the desired antigen. Some amino acids in the framework are chosen to be the same as the amino acids at those positions in the donor rather than in the acceptor. Specifically, these documents describe the preparation of a humanized antibody that binds to a receptor by combining the CDRs of a mouse monoclonal antibody with human immunoglobulin framework and constant regions. Human framework regions can be chosen to maximize homology with the mouse sequence. A computer model can be used to identify amino acids in the framework region which are likely to interact with the CDRs or the specific antigen and then mouse amino acids can be used at these positions to create the humanized antibody.

The above-cited documents also propose four possible criteria, which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3 angstrom units of the CDRs in a three dimensional model of the antibody and is predicted to be capable of interacting with the CDRs. The above methods are merely illustrative of some of the methods that one skilled in the art could employ to make humanized antibodies.

This invention provides an isolated nucleic acid molecule encoding the above antibody. The nucleic acid molecule includes but is not limited to RNA, genomic DNA and cDNA.

This invention provides a labeled antibody. The antibody may be labeled with a detectable marker including, but not limited to: a radioactive marker, a colorimetric marker, a luminescent marker, an enzyme marker, a fluorescent marker, or gold. Methods for attaching markers to antibodies are well known in the art.

Radioactive markers include, but are not limited to: ³H, ¹⁴C, ³²P, ³³P, ³⁵S, ³⁶Cl, ⁵¹Cr, ⁵⁷Co, ⁵⁹Co, ⁵⁹Fe, ⁹⁰Y, ¹²⁵I, ¹³¹I, and ¹⁸⁶Re.

Fluorescent markers include, but are not limited to, fluorescein, rhodamine and auramine, isothiocyanate, rhodamine, phycoerythrin, phycocyanin, allophycocyanin, o-phthaldehyde, fluorescamine and Texas Red.

Colorimetric markers include, but are not limited to, biotin and digoxigenin.

Additionally, chemiluminescent compounds may be used as markers. Typical chemiluminescent compounds include, but are not limited to, luminol, isoluminol, aromatic acridinium esters, imidazoles, acridinium salts, and oxalate esters. Similarly, bioluminescent compounds may be utilized as markers, the bioluminescent compounds including luciferin, luciferase, and aequorin.

Enzyme markers include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

The antibody can also be labeled using fluorescence emitting metals such as ¹⁵²Eu, or other of the lanthanide series. These metals can be attached to the antibody using such metal groups as diethylenetriamine pentacetic acid (EDTA).

Once labeled, the antibody may be employed to identify and quantify the human BSEP polypeptide variants of the invention utilizing techniques well known to the art. See, for example, Immunology Methods Manual, ed. by Lefkovitis, Academic Press, 1996.

Methods of Detecting BSEP Polypeptide Variants

The invention also provides methods for detecting a BSEP polypeptide variant in a sample comprising: (a) contacting a suitable sample with a compound that specifically binds to the BSEP polypeptide variant; and (b) determining whether any compound is bound to the BSEP polypeptide variant, where the presence of any compound bound to the BSEP polypeptide variant detects the BSEP polypeptide variant in the sample. In one embodiment, any unbound compound is removed prior to determining whether any compound is bound to the BSEP polypeptide variant. In one embodiment, the BSEP polypeptide variant is an insert variant.

In one embodiment the compound is immobilized on a solid support. In one embodiment the solid support is a microtiter plate well. In another embodiment, the solid support is a membrane such as a cellulose membrane, a nitrocellulose membrane, or a nylon membrane. In another embodiment, the solid support is a bead. In a further embodiment, the solid support is a surface plasmon resonance sensor chip. The surface plasmon resonance sensor chip can have pre-immobilized streptavidin. In one embodiment, the surface plasmon resonance sensor chip is a BIAcore™ chip (Biacore, Piscataway, N.J.).

The term “suitable sample,” as it refers to samples used for detecting polypeptides include, but is not limited to: cells, cell lysates, protein or membrane extracts of cells, body fluids, or tissue samples.

In one embodiment of the method described immediately above, the compound capable of specifically binding the BSEP polypeptide variant is an antibody. In one embodiment the antibody specifically binds to the human BSEP polypeptide insert variant of SEQ ID NO:4. In another embodiment, the antibody specifically binds to the human BSEP polypeptide insert variant of SEQ ID NO:8.

In one embodiment the compound capable of specifically binding the BSEP polypeptide variant is labeled with a detectable marker. In one embodiment, the detectable marker is a radioactive marker, a colorimetric marker, a luminescent marker, enzyme marker, or a fluorescent marker. When a compound is labeled with a detectable marker, the compound, and anything bound to the compound, can be detected by detecting the labeled marker. Methods for attaching markers to compounds are well known in the art.

As used herein “determining whether any compound is bound to BSEP polypeptide variant” can be done by any of the methods known to those skilled in the art.

Methods such as surface plasmon resonance may be used to measure the direct binding of the compound to the BSEP polypeptide variant using commercially available instruments, methods and reagents (Biacore, Piscataway, N.J.). Other methods that can be used to measure the binding of the compound to the BSEP polypeptide variant include, but are not limited to, Western blot, immunoprecipitation and ELISA.

When the compound capable of specifically binding the BSEP polypeptide variant is an antibody, “determining whether the compound is bound to BSEP polypeptide variant” can be accomplished using any of a variety of immunoassays, such as RIA (radioimmunoassay), ELISA, Western blot analysis and immunoprecipitation.

Methods of Detecting Nucleic Acids Encoding BSEP Polypeptide Variants

The invention also provides methods for detecting a nucleic acid which encodes a BSEP polypeptide variant comprising: (a) contacting a suitable sample with a compound capable of specifically binding a nucleic acid encoding a BSEP polypeptide variant; and (b) determining whether any compound is bound to the nucleic acid, where the presence of compound bound to the nucleic acid in the sample. In one embodiment, any unbound compound is removed prior to determining whether the compound is removed prior to determining whether any compound is bound to the nucleic acid. In one embodiment, the BSEP polypeptide variant is an insert variant.

In one embodiment the compound is immobilized on a solid support. In one embodiment the solid support is a microtiter plate well. In another embodiment, the solid support is a membrane such as a cellulose membrane, a nitrocellulose membrane, or a nylon membrane. In another embodiment, the solid support is a bead. In a further embodiment, the solid support is a surface plasmon resonance sensor chip. The surface plasmon resonance sensor chip can have pre-immobilized streptavidin. In one embodiment, the surface plasmon resonance sensor chip is a BIAcore™ chip.

The term “suitable sample,” as it refers to samples used for detecting nucleic acids includes, but is not limited to, cells, cell lysates, nucleic acids extracts of cells, tissue samples, or body fluids. Body fluids include, but are not limited to, blood, serum and saliva.

The term “specifically binding to a nucleic acid,” as used herein, refers to a binding reaction that is determinative of the presence of the nucleic acid in a population of nucleic acids or other material.

In one embodiment of the method described immediately above, the compound is a nucleic acid.

In one embodiment, the compound is a nucleic acid complementary to a nucleic acid encoding SEQ ID NO:6 or a fragment thereof. In one embodiment, the compound is a nucleic acid complementary to SEQ ID NO:5 or a fragment thereof. In one embodiment, the compound is a nucleic acid complementary to a nucleic acid encoding SEQ ID NO:10 or a fragment thereof. In one embodiment, the compound is a nucleic acid complementary to SEQ ID NO:9 or a fragment thereof. In one embodiment, the compound is a nucleic acid complementary to a nucleic acid encoding SEQ ID NO:11 or a fragment thereof. In one embodiment, the compound is a nucleic acid complementary to a SEQ ID NO:12 or a fragment thereof.

In one embodiment the compound capable of specifically binding the nucleic acid encoding the BSEP polypeptide variant is labeled with a detectable marker. In one embodiment, the detectable marker is a radioactive label, a calorimetric marker, a luminescent marker, enzyme marker or a fluorescent marker. When a compound is labeled with a detectable marker, the compound, and anything bound to the compound, can be detected by detecting the labeled marker. Methods for attaching markers to compounds are well known in the art.

As used herein “determining whether any compound is bound to nucleic acid” can be done by any of the methods known to those skilled in the art. These methods include, but are not limited to, Southern blotting, Northern blotting and RNA protection assays.

Methods of Diagnosis

The present invention also provides methods of determining whether a subject is expressing a BSEP polypeptide variant comprising: (a) contacting a suitable sample from the subject with a compound that specifically binds to the BSEP polypeptide variant; and (b) determining whether any compound is bound to the BSEP polypeptide variant, wherein the presence of the compound bound to the BSEP polypeptide variant indicates that the individual is expressing the BSEP polypeptide variant. In one embodiment, any unbound compound is removed prior to determining whether any compound is bound to the BSEP polypeptide variant. In one embodiment, the BSEP polypeptide variant is a BSEP polypeptide insert variant.

A “suitable sample” in connection to the above method of determining whether a subject is expressing a human BSEP polypeptide variant refers to any sample from the subject that could contain the BSEP polypeptide variant. Examples include, but are not limited to, body fluids and tissue samples. Examples of body fluids include, but are not limited to, blood, serum, urine and saliva.

In one embodiment the compound is an antibody that specifically binds to the BSEP polypeptide insert variant of SEQ ID NO:4. In another embodiment the compound is an antibody that specifically binds to the BSEP polypeptide insert variant of SEQ ID NO:8.

Methods of determining whether the compound is bound to the BSEP polypeptide variant are well known to a person having ordinary skill in the art. These methods include, but are not limited to, Western blot, immunoprecipitation and ELISA.

A “subject,” as used herein means any animal or artificially modified animal expressing a BSEP polypeptide variant. The animals include but are not limited to mice, rats, dogs, guinea pigs, ferrets, rabbits, humans, and other primates.

Methods of Detecting Nucleic Acids Encoding BSEP Polypeptide Variants Using RT-PCR or PCR

The invention also provides methods of detecting a nucleic acid which encodes a BSEP polypeptide variant comprising: (a) obtaining cDNA from mRNA obtained from a suitable sample; (b) amplifying the cDNA corresponding to the nucleic acid encoding the BSEP polypeptide or a portion of said nucleic acid; (c) comparing the amplified cDNA to the DNA of a nucleic acid known to encode a BSEP polypeptide, wherein a difference between the amplified cDNA and the DNA from a nucleic acid known to encode a BSEP polypeptide indicates the detection of a nucleic acid encoding a BSEP polypeptide variant in the sample.

The invention also provides methods for detecting a nucleic acid which encodes a BSEP polypeptide variant comprising: (a) contacting a suitable sample with a compound capable of specifically binding a nucleic acid encoding a BSEP polypeptide variant; and (b) determining whether any compound is bound to the nucleic acid, where the presence of compound bound to the nucleic acid in the sample.

In one embodiment, the BSEP polypeptide variant is a human BSEP polypeptide variant. In one embodiment, the BSEP polypeptide variant is an insert variant.

As used herein, “mRNA” refers to messenger RNA. As used herein, “cDNA” refers to complementary DNA.

The term “suitable sample,” as it refers to samples used for detecting nucleic acids includes, but is not limited to, cells, cell lysates, nucleic acids extracts of cells, tissue samples, or body fluids. Body fluids include, but are not limited to, blood, serum and saliva. In one embodiment, the suitable sample is obtained from a subject.

Methods of obtaining mRNA from a suitable sample are well known in the art. Further, methods of making cDNA from mRNA, such as reverse transcription, are also well known in the art.

As used herein, “amplifying” means increasing the numbers of copies of a specific DNA fragment. In one embodiment, the amplifying of the cDNA is carried out using PCR (polymerase chain reaction).

In one embodiment, the amplifying of the cDNA is accomplished using primers flanking the entire reading frame of a gene encoding a BSEP polypeptide are used to amplify the cDNA. In another embodiment, the amplifying of the cDNA is accomplished out using primers flanking a portion of a nucleic acid encoding a BSEP polypeptide.

If the BSEP polypeptide variant is an insert variant, the amplifying of the cDNA may be accomplished using primers that flank the inserted sequence. In another embodiment, the primers used flank a portion of the cDNA which portion comprises the inserted sequence, but which is bigger than the inserted sequence. In one embodiment, the primers used flank the entire open reading frame a nucleic acid encoding the BSEP polypeptide variant.

In one embodiment, comparing the amplified cDNA to the DNA of a nucleic acid known to encode a BSEP polypeptide is accomplished by comparing the sequence of the amplified cDNA to the sequence of a nucleic acid known to encode a BSEP polypeptide. The presence of additional nucleotides in the BSEP polypeptide variant will indicate that the BSEP polypeptide variant has an insertion.

In another embodiment, comparing the amplified cDNA to the DNA of a nucleic acid known to encode a BSEP polypeptide is accomplished by comparing the size of the amplified cDNA to the size of the DNA of a gene known to encode a BSEP polypeptide. A difference in size will indicate that the amplified DNA encodes a BSEP polypeptide variant. For example, if the amplified DNA encodes more than 1321 amino acids, then the amplified DNA encodes a BSEP polypeptide insert variant.

Methods of Diagnosis Using RT-PCR

The invention also provides methods of determining whether a subject is expressing a BSEP polypeptide insert variant comprising: (a) obtaining cDNA from mRNA obtained from a suitable sample from the subject; (b) amplifying the cDNA corresponding to the insertion of the BSEP polypeptide insert variant or a portion of said insertion; and (c) comparing the amplified cDNA to the DNA of a nucleic acid known to encode a BSEP polypeptide, wherein the presence of an insertion in the amplified cDNA indicates that the subject is expressing the BSEP polypeptide insert variant.

A “suitable sample” in connection to the above method of determining whether a whether a subject is expressing a human BSEP polypeptide variant refers to any sample from the subject that could contain the BSEP polypeptide variant. Examples include, but are not limited to, body fluids and tissue samples. Examples of body fluids include, but are not limited to, blood, serum, urine and saliva.

In one embodiment, the amplifying of the cDNA may be accomplished using primers that flank the inserted sequence. In another embodiment, the primers used flank a bigger portion of the cDNA which portion comprises the inserted sequence. In one embodiment, the primers used flank the entire open reading frame of a nucleic acid encoding the BSEP polypeptide variant.

In one embodiment, comparing the amplified cDNA to the DNA of a nucleic acid known to encode a BSEP polypeptide is accomplished by comparing the sequence of the amplified cDNA to the sequence of a nucleic acid known to encode a BSEP polypeptide. The presence of additional nucleotides in the BSEP polypeptide variant will indicate that the BSEP polypeptide variant has an insertion, and is a BSEP polypeptide insert variant.

In another embodiment, comparing the amplified cDNA to the DNA of a nucleic acid known to encode a BSEP polypeptide is accomplished by comparing the size of the amplified cDNA to the size of the DNA of a gene known to encode a BSEP polypeptide. A difference in size will indicate that the amplified DNA encodes a BSEP polypeptide variant.

Methods of Identifying BSEP Substrates or Modulators

The present invention provides methods of identifying compounds that are substrates and/or modulators of the BSEP polypeptide variants of the invention. Compounds that are substrates and/or modulators of BSEP polypeptide variants could be potentially toxic to cells. Therefore, identifying compounds that are substrates and/or modulators of the BSEP polypeptide variants of the invention could be useful in drug screening, to identify compounds that could be potentially toxic to a subject expressing a BSEP polypeptide variant.

The term “compound,” as used herein, refers to can be any chemical or biological agent. Some examples of such test agents are synthetic chemicals, naturally occurring chemicals, proteins (e.g., antibodies), nucleotides (e.g., antisense oligonucleotides, interference RNA oligonucleotides), etc.

The term “modulator,” as used herein, refers to a compound that alters the function or activity of a BSEP polypeptide variant. The “modulator” can be an inhibitor, an activator, or an inducer a BSEP polypeptide variant, or a combination thereof. An “inhibitor” is a compound that can inhibit or decrease the transport activity of the BSEP polypeptide variant. An “activator” is a compound that can increase the transport activity of the BSEP polypeptide variant. An “inducer” is a compound that can increase the expression of the BSEP polypeptide variant, therefore increasing the transport activity of the BSEP polypeptide variant.

The term “substrate,” as used herein with respect to a human BSEP polypeptide variant refers to molecule that is to be transported by a human BSEP polypeptide variant. Some examples of such transport activities are efflux from inside cells to outside cells, or uptake into inside-out membrane vesicles. The substrate of a BSEP polypeptide variant can be any compound capable of being transported by the BSEP polypeptide variant. In one embodiment, the substrate of a BSEP polypeptide variant is any bile acid or salt thereof, or conjugate thereof. In one embodiment, the substrate of a BSEP polypeptide variant is selected from the group consisting of: cholyltaurine, taurocholate, taurochenodeoxycholate, taurodeoxycholate, tauroursodeoxycholate, glycohenodeoxycholate, glycocholate, lithocholate, deoxycholate, chenodeoxycholate, cholyl lysyl fluorescin AM, cholyl lysyl fluorescin diacetate, and calcein-AM. In another embodiment the substrate of a BSEP polypeptide variant is a compound identified using the methods described herein.

Binding Assays

The present invention provides methods for determining whether a compound is a substrate or modulator of a BSEP polypeptide variant comprising: (a) contacting the compound with a BSEP polypeptide variant and (b) determining whether any compound binds to the BSEP polypeptide variant, wherein the presence of compound bound to the BSEP polypeptide variant indicates that the compound is a substrate or modulator of the BSEP polypeptide variant. In one embodiment, step (a) is carried out under conditions permitting binding of the compound to the BSEP polypeptide variant.

In one embodiment, the BSEP polypeptide variant is a BSEP polypeptide insert variant. In one embodiment, the BSEP polypeptide variant is isolated or purified. In another embodiment, the BSEP polypeptide variant is in a cell that naturally contains the BSEP polypeptide variant. In another embodiment, the BSEP polypeptide variant is in a cell that has been genetically engineered to express a BSEP polypeptide variant. In another embodiment, the BSEP polypeptide variant is in a cell membrane preparation. In another embodiment, the BSEP polypeptide variant is in a cell membrane vesicle or an inside-out cell membrane vesicle.

In one embodiment, the BSEP polypeptide variant is immobilized on a solid support. In one embodiment of the method described herein, the solid support is a microtiter plate well. In another embodiment, the solid support is a membrane such as a cellulose membrane, a nitrocellulose membrane, or a nylon membrane. In another embodiment, the solid support is a bead. In a further embodiment, the solid support is a surface plasmon resonance sensor chip. The surface plasmon resonance sensor chip can have pre-immobilized streptavidin. In one embodiment, the surface plasmon resonance sensor chip is a BIAcore™ chip.

As used herein, a “cell membrane preparation” refers to isolated or reconstituted cell membranes. In one embodiment, the cell membrane preparation is a cell membrane vesicle. In another embodiment, the cell membrane preparation is an inside-out cell membrane vesicle.

As used herein, a “cell membrane vesicle” refers to a closed membrane shell. As used here in, “inside-out cell membrane vesicle” refers to a closed membrane shell where the natural topography of the membrane is inverted. A person of ordinary skill in the art would know how to prepare cell membrane vesicles and inside-out cell membrane vesicles.

The amount of compound bound to the BSEP polypeptide variant can be measured using any method known in the art. In one embodiment, the amount of compound bound to the BSEP polypeptide variant can be determined by measuring the concentration of unbound or free compound, and comparing this concentration to the concentration of compound originally added. If binding occurs between the compound and the BSEP polypeptide variant, the concentration of unbound or free compound will be less than the concentration of compound originally added.

In one embodiment the compound is labeled with a detectable marker. The detectable marker can be, but is not limited to, a radioactive label, a colorimetric marker, a luminescent marker, enzymatic marker, a fluorescent marker or a marker that is capable of emitting electromagnetic energy.

In one embodiment, this method will be carried out using a high throughput assay. “High throughput assay” as used herein refers to an assay, which provides for multiple candidate agents or samples to be screened simultaneously. As further described below, examples of such assays may include the use of microtiter plates which are especially convenient because a large number of assays can be carried out simultaneously, using relatively small amounts of reagents and samples.

Competition Binding Assays

The present invention also provides s methods for determining whether a compound is a substrate or modulator of a BSEP polypeptide variant comprising: (a) contacting the BSEP polypeptide variant with a known binder and the compound; (b) measuring the amount of binder bound to the BSEP polypeptide variant; and (c) comparing the amount of binder bound to the BSEP polypeptide variant in step (b) with the amount of binder bound to the BSEP polypeptide variant in the absence of the compound, and determining whether the presence of the compound increased or decreased the amount of binder bound to the BSEP polypeptide variant, wherein an increase or a decrease in the amount of binder bound to the BSEP polypeptide variants indicates that the compound is a substrate or modulator of BSEP. In one embodiment, step (a) is carried out under conditions permitting binding of the known binder to the BSEP polypeptide variant.

The invention also provides methods for determining whether a compound is a substrate or modulator of a BSEP polypeptide variant comprising: (a) contacting the BSEP polypeptide variant with the compound and a known binder of the BSEP polypeptide variant; (b) measuring the amount of compound bound to the BSEP polypeptide variant; and (c) comparing the amount of binding in step (b) with the amount of binding in the absence of the known binder, and determining whether the presence of the known binder increased or decreased the binding of the compound to the BSEP polypeptide variant, wherein an increase or a decrease in the amount of compound bound to the BSEP polypeptide variants indicates that the compound is a substrate or modulator of BSEP. In one embodiment, step (a) is carried out under conditions permitting binding of the known binder to the BSEP polypeptide variant.

In one embodiment, the BSEP polypeptide variant is a BSEP polypeptide insert variant. In one embodiment, the BSEP polypeptide variant is isolated or purified. In another embodiment, the BSEP polypeptide variant is in a cell that naturally contains the BSEP polypeptide variant. In another embodiment, the BSEP polypeptide variant is in a cell that has been genetically engineered to express a BSEP polypeptide variant. In another embodiment, the BSEP polypeptide variant is in a cell membrane vesicle or an inside-out cell membrane vesicle. In another embodiment, the BSEP polypeptide variant is in a cell membrane preparation.

In one embodiment, the BSEP polypeptide variant is immobilized on a solid support. In one embodiment of the method described herein, the solid support is a microtiter plate well. In another embodiment, the solid support is a membrane such as a cellulose membrane, a nitrocellulose membrane, or a nylon membrane. In another embodiment, the solid support is a bead. In a further embodiment, the solid support is a surface plasmon resonance sensor chip. The surface plasmon resonance sensor chip can have pre-immobilized streptavidin. In one embodiment, the surface plasmon resonance sensor chip is a BIAcore™ chip.

As used herein, a “known binder,” refers to a compound that is known to bind to the BSEP polypeptide variant. In one embodiment, the known binder is selected from the group consisting of: cholyltaurine, taurocholate, taurochenodeoxycholate, taurodeoxycholate, tauroursodeoxycholate, glycohenodeoxycholate, glycocholate, lithocholate, deoxycholate, chenodeoxycholate, cholyl lysyl fluorescin AM, cholyl lysyl fluorescin diacetate, and calcein-AM. In another embodiment, the known binder is a binder identified in any of the methods described herein.

In one embodiment the compound and the known binder are introduced at the same time. In another embodiment, the known binder is introduced first and then the compound is added. In another embodiment, the compound is introduced first and then the known binder is added.

Determining whether a compound binds the BSEP polypeptide variant can be accomplished using any method known in the art including, but not limited to, equilibrium dialysis and rapid filtration.

The amount of compound or known binder bound to the BSEP polypeptide variant can be measured using any method known in the art. In one embodiment, the amount of compound or known binder bound to the BSEP polypeptide variant can be determined by measuring the concentration of unbound or free compound or known binder, and comparing this concentration to the concentration of compound or known binder originally added. If binding occurs between the compound or known binder and the BSEP polypeptide variant, the concentration of unbound or free compound or known binder will be less than the concentration of compound or known binder originally added.

In one embodiment, the compound is labeled with a detectable marker. In another embodiment, the known binder is labeled with a detectable marker. The detectable marker can be, but is not limited to, a radioactive label, a colorimetric marker, a luminescent marker, enzymatic marker, a fluorescent marker or a marker that is capable of emitting electromagnetic energy.

In one embodiment, invention methods may be carried out using high throughput assays. A high throughput assay for measuring the unbound or free concentration of a compound has been disclosed, for example, in European Patent EP1088589 (Banker M J et al.). However, those skilled in the art are knowledgeable about many other high throughput assays that might be applied to the methods of the present invention.

ATPase Assays

Where a compound is a substrate of the BSEP polypeptide variant, ATP will be hydrolyzed. The energy derived from this reaction is used for the transport activity of the BSEP polypeptide variant. Therefore, a measure of the ATPase activity can be used to identify substrates and/or modulators of the BSEP polypeptide variants.

The invention also provides methods for determining whether a compound is a substrate of a BSEP polypeptide variant comprising: (a) contacting the compound with the BSEP polypeptide variant and ATP; and (b) detecting the ATPase activity of the BSEP polypeptide insert variant, wherein the detection of ATPase activity indicates that the compound is a substrate of the BSEP polypeptide variant.

The invention also provides methods for determining whether a compound is an modulator of a BSEP polypeptide variant comprising: (a) contacting the BSEP polypeptide variant with a substrate of the BSEP polypeptide variant, a compound and ATP; (b) measuring the ATPase activity of the BSEP polypeptide variant; and (c) comparing the ATPase activity of the BSEP polypeptide variant in step (b) with the ATPase activity of the BSEP polypeptide variant in the absence of the compound, and determining whether the compound modulated the ATPase activity of the BSEP polypeptide variant, wherein an increase or a decrease in the ATPase activity indicates that the compound is a substrate or modulator of the BSEP polypeptide variant.

Where the compound is an activator or inducer of the BSEP polypeptide variant, the ATPase activity will increase in the presence of the compound. Where the compound is an inhibitor of the BSEP polypeptide variant, the ATPase activity will decrease in the presence of the compound.

In one embodiment, the BSEP polypeptide variant is a BSEP polypeptide insert variant. In another embodiment, the BSEP polypeptide variant is in a cell that naturally contains the BSEP polypeptide variant. In another embodiment, the BSEP polypeptide variant is in a cell membrane vesicle or an inside-out cell membrane vesicle. In another embodiment, the BSEP polypeptide variant is in a cell membrane preparation. In one embodiment, the BSEP polypeptide variant is isolated. In one embodiment, the BSEP polypeptide variant is purified.

The term “ATPase activity,” as used herein, refers to the ability of the polypeptide to hydrolyze ATP. The hydrolysis of ATP can be measured by either a decrease in chemical reactants (ATP), or an increase in chemical products (ADP and phosphate). Methods of detecting or measuring ATPase activity are well known in the art. An ATPase assay for a human transporter is disclosed in Shimabuku et al., J. Biol. Chem. 267(7):4308-11 (1992). A higher throughput ATPase assay was published in Szabo et al., J. Biol. Chem. 273(17):10132-38 (1998).

Transport Assays

The present invention also provides methods for determining whether a compound is a substrate of BSEP comprising: (a) contacting the compound with the BSEP polypeptide variant; (b) determining whether the compound is transported by the BSEP polypeptide variant, wherein the transport of the compound by the BSEP polypeptide variant indicates that the compound is a substrate of the BSEP polypeptide variant.

Determining whether the compound is transported by the BSEP polypeptide variant can be done using a variety of methods known to those skilled in the art. See, for example, Byrne, J. A., et al., Gastroenterology 123(5):1649-58 (2002); Gerloff, T., et al., European Journal of Biochemistry 269(14):3495-503 (2002); Bode, K. A., et al., Biochemical Pharmacology 64(1):151-58 (2002); Noe, J., et al., Hepatology 33(5):1223-31 (2001); Kullak-Ublick, G. A., et al., Seminars in Liver Disease 20(3):273-92 (2000); Stieger, B., et al., Gastroenterology 118(2):422-30 (2000); Ballatori, N., et al., American Journal of Physiology—Gastrointestinal & Liver Physiology 278(1):G57-63 (2000).

In one embodiment, the BSEP polypeptide variant is a BSEP polypeptide insert variant. In another embodiment, the BSEP polypeptide variant is in a cell that has been genetically engineered to express a BSEP polypeptide variant. In one embodiment, the cells that contain BSEP polypeptide variants can be grown in culture, either in suspension, or attached to a surface (e.g., plastics or glass or porous surface). In another embodiment, the cells are grown on a porous surface such that a compound can be added to the basal side of the cells and efflux of the compound can be directed to the apical surface where the BSEP polypeptide variants are located.

In another embodiment, the BSEP polypeptide variant is in a cell membrane preparation. In another embodiment, the BSEP polypeptide variant is in a cell membrane vesicle or an inside-out cell membrane vesicle.

In another embodiment, the BSEP polypeptide variant is isolated. In another embodiment, the BSEP polypeptide variant is purified.

The “transport” or “transport activity” of a BSEP polypeptide or a BSEP polypeptide variant refers to the ability of a BSEP polypeptide to transport a compound from the inside a cell to the outside a cell or cell membrane vesicle, or from the outside of an “inside-out” cell membrane vesicle to the inside of a cell membrane vesicle.

In one embodiment, the compound is labeled with a detectable marker. The detectable marker can be, but is not limited to, a radioactive label, a calorimetric marker, a luminescent marker, enzymatic marker, a fluorescent marker or a marker that is capable of emitting electromagnetic energy.

In one embodiment, the method will be carried out using high throughput assay.

In one embodiment, the transport activity can be detected by measuring either the intracellular concentration of compound over time, or the extracellular concentration of compound over time, or both. If the compound is a substrate of the BSEP polypeptide variant and the assay is carried out in a cell, the intracellular concentration of the compound will decrease over time and the extracellular concentration of the compound will increase over time. If the compound is a substrate of the BSEP polypeptide variant and the assay is carried out in an inside-out membrane vesicle, the compound will accumulate inside the inside-out cell membrane vesicle over time, and the concentration of the compound outside of the inside-out cell membrane vesicle will decrease over time.

Competition Transport Assays

The invention also relates to methods for determining whether a compound is a substrate or modulator of a human BSEP polypeptide variant comprising: (a) contacting the compound with the BSEP polypeptide variant and a known substrate of the BSEP polypeptide variant; (b) measuring the activity of the BSEP polypeptide variant with respect to the known substrate; and (c) comparing the activity of the BSEP polypeptide variant with respect to the known substrate in step (b) with the activity of the BSEP polypeptide with respect to the known substrate in the absence of the compound, wherein an increase or a decrease in transport of activity of the BSEP polypeptide insert variant with respect to the known substrate indicates that the compound is a substrate or modulator of the BSEP polypeptide insert variant. In one embodiment, the BSEP polypeptide variant is a BSEP polypeptide insert variant.

In one embodiment, the BSEP polypeptide variant is in a cell that naturally contains the BSEP polypeptide variant. In another embodiment, the BSEP polypeptide variant is in a cell that has been genetically engineered to express a BSEP polypeptide variant. In one embodiment, the cells that contain BSEP polypeptide variants can be grown in culture, either in suspension, or attached to a surface (e.g., plastics or glass or porous surface). In another embodiment, the cells are grown on a porous surface such that a compound can be added to the basal side of the cells and efflux of the compound can be directed to the apical surface where the BSEP polypeptide variants are located.

In another embodiment, the BSEP polypeptide variant is in a cell membrane vesicle, or an inside-out cell membrane vesicle.

The “transport activity” of a BSEP polypeptide or a BSEP polypeptide variant refers to the ability of a BSEP polypeptide to transport a compound from the inside a cell to the outside a cell or cell membrane vesicle, or from the outside of an “inside-out” cell membrane vesicle to the inside of a cell membrane vesicle.

In one embodiment the substrate is labeled with a detectable marker. The detectable marker can include, but is not limited to, a radioactive label, a colorimetric marker, a luminescent marker, enzymatic marker, a fluorescent marker or a marker that is capable of emitting electromagnetic energy.

The known substrate can be any substrate of the BSEP polypeptide variant. In one embodiment, the known substrate is selected from the group consisting of: cholyltaurine, taurocholate, taurochenodeoxycholate, taurodeoxycholate, tauroursodeoxycholate, glycohenodeoxycholate, glycocholate, lithocholate, deoxycholate, chenodeoxycholate, cholyl lysyl fluorescin AM, cholyl lysyl fluorescin diacetate, and calcein-AM. In another embodiment, the known substrate is a substrate identified in any of the methods described herein.

In one embodiment the known substrate can be added prior to the compound. In another embodiment, the known substrate is added simultaneously with the compound. In another embodiment, the known substrate is added after the compound.

In one embodiment, the method will be carried out using a high throughput assay.

In one embodiment, the transport activity can be detected by measuring either the intracellular concentration of compound over time, or the extracellular concentration of compound over time, or both.

If the compound is an inhibitor of the BSEP polypeptide variant, the transport activity of the BSEP polypeptide variant will decrease. If the compound is an activator or inducer of the BSEP polypeptide variant, the transport activity of the BSEP polypeptide variant will increase. By comparing the transport activity of the BSEP polypeptide variant in the presence and in the absence of the compound, the degree of inhibition, activation or inducement by the compound can be determined.

A method of determining the transport activity of a BSEP polypeptide, wherein the BSEP polypeptide is expressed in a host cell is described in Lecureur et al., Molecular Pharmacology, 57:24-35 (2000).

A method of determining the transport activity of a BSEP polypeptide, wherein the BSEP polypeptide is on a cell membrane vesicle, is described in Stieger et al., Gastroenterology, 118:422-30, (2000).

All of the methods disclosed herein to identify activators are similarly expected to also identify inducers. However, as presently understood, the induction process usually takes a much longer time than the activation process. Therefore, to test whether a compound is an inducer of the polypeptide variant, the compound is preferably introduced to the assay system hours or even days before the known substrate is added. In addition, there are additional assay formats that can be used to unambiguously identify a compound as an inducer. By definition, an inducer will increase messenger RNA and protein levels of BSEP polypeptide variant. Therefore, any of the diagnostic methods to measure the level of messenger RNA, such as quantitative PCR, Invader®) assay (Third Wave Technologies, Madison, Wis.), Northern blots and RNAse protection assays, and protein levels of the BSEP polypeptide variants, such as Western blots, ELISA assays, cytoimmunofluorescence assays, can be used to identify whether a test agent is an inducer of the BSEP polypeptide variants.

All of the method of identifying compounds that are substrates and/or modulators of the BSEP polypeptide variants of the invention can be carried out in 24-well or higher density microarrays or microwells to enable higher assay throughput. In an embodiment where the compound or substrate is labeled with a fluorescent marker, the binding and the transport process of such molecules can be detected and measured automatically by quantitative fluorometry (e.g., automated fluorescence plate readers, automated and quantitative fluorescence microscope, etc.). Examples of automated fluorescence plate readers that are capable of performing kinetic measurements are: Victor V from Perkin Elmer, FLIPR HTS reader from Molecular Devices, et al. Examples of the automated and quantitative fluorescence microscopes that are capable of performing kinetic measurements are: ArrayScan® Kinetic Scan™ (Cellomics, Pittsburgh, Pa.), Pathway HT™ system (Atto Bioscience, Rockville, Md.), and the like. The combination of the disclosed assay methods and modern day automated equipments is expected to deliver unprecedented capacity and throughput to identify substrates, inhibitors, activators, and inducers of human BSEP polypeptide variants.

In one embodiment, any of the methods of identifying substrates and/or modulators of a BSEP polypeptide variant described herein can be used to characterize the transport function of the claimed BSEP polypeptide insert variants as compared to previously known human BSEP polypeptides. An increase or decrease in the transport activity of the BSEP polypeptide variants may be used to explain decreased or increased bile salt efflux in humans having the BSEP polypeptide variants.

Methods of Inhibiting the Expression of a Human BSEP Polypeptide Variant

The invention also provides methods of inhibiting the expression of a BSEP polypeptide variant in a cell comprising contacting the cell with a compound capable of inhibiting the expression of the BSEP polypeptide variant. In one embodiment the BSEP polypeptide variant is a human BSEP polypeptide insert variant.

In one embodiment the method will be carried out in vitro. In one embodiment the method will be carried out in vivo. These methods could be used in research, diagnosis and treatment.

In research, these methods could be used, for example, to elucidate the function of a BSEP polypeptide variant of the invention with respect to a human BSEP polypeptide or another BSEP polypeptide variant.

In one embodiment, the compound capable of inhibiting the expression of a BSEP polypeptide variant is an antisense oligonucleotide capable of specifically hybridizing to the BSEP polypeptide variant.

It is understood in the art that the sequence of an antisense compound need not be 100% complementary to that of its target nucleic acid to specifically hybridize to that sequence. An antisense compound specifically hybridizes to a target DNA or RNA sequence when binding of the compound to the target DNA or RNA sequence interferes with the normal function of the target DNA or RNA. This interference should cause a loss of utility, and there should be a sufficient degree of complementarity to avoid non-specific binding of the antisense compound to non-target sequences under conditions in which specific binding is desired, i.e., under physiological conditions in the case of in vivo assays or therapeutic treatment, and in case of in vitro assays, under conditions in which the assays are performed.

An “oligonucleotide,” as used herein, refers to an oligomer or polymer of a ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

The sequence of an antisense oligonucleotide capable of specifically hybridizing to a BSEP polypeptide variant can be identified through routine experimentation. In one embodiment the antisense oligonucleotide is capable of specifically hybridizing to a nucleic acid sequence from the group consisting of: (a) a nucleic acid encoding SEQ ID NO:6 or a fragment thereof; (b) SEQ ID NO:5 or a fragment thereof; (c) a nucleic acid encoding SEQ ID NO:10 or a fragment thereof; (d) SEQ ID NO:9 or a fragment thereof; (e) a nucleic acid encoding SEQ ID NO:11 or a fragment thereof; and (f) SEQ ID NO:12 or a fragment thereof.

In another embodiment, the compound capable of inhibiting the expression of a BSEP polypeptide variant is an RNAi construct. In one embodiment the RNAi construct is capable of specifically hybridizing to a nucleic acid sequence selected from the group consisting of: (a) a nucleic acid encoding SEQ ID NO:6 or a fragment thereof; (b) SEQ ID NO:5 or a fragment thereof; (c) a nucleic acid encoding SEQ ID NO:10 or a fragment thereof; (d) SEQ ID NO:9 or a fragment thereof; (e) a nucleic acid encoding SEQ ID NO:11 or a fragment thereof; and (f) SEQ ID NO:12 or a fragment thereof.

The antisense oligonucleotides and RNAi constructs can be used to specifically inhibit the expression of the claimed human BSEP polypeptide variants without inhibiting the human BSEP polypeptide. Using this technology, the specific function of each claimed human BSEP polypeptide variant can be studied. Further, antisense oligonucleotides and RNAi constructs may be used for disease treatment.

Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability. See, for example, Antisense Technology in Methods in Enzymology, Vols. 313-314, ed. by Phillips, Abelson and Simon, Academic Press, 1999.

Without being bound by any particular theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the inactivation (at least partially) of the mRNA encoding a specific protein. Accordingly, antisense oligonucleotides decrease the translation of a particular protein.

The oligonucleotides can be DNA or RNA, or chimeric mixtures or derivatives or modified versions thereof, single-stranded or double-stranded. The oligonucleotide can be modified at the base moiety, sugar moiety, or phosphate backbone, for example, to improve its stability, hybridization, etc. The oligonucleotide may include other appended groups such as peptides (e.g., for targeting host cell receptors), or agents facilitating transport across the cell membrane (see, e.g., Letsinger et al., Proc. Natl. Acad. Sci. U.S.A. 86:6553-56 (1989); Lemaitre et al., Proc. Natl. Acad. Sci. U.S.A. 84:648-52 (1987); International Patent Publication No. WO88/09810) or the blood-brain barrier (see, e.g., International Patent Publication No. WO89/10134), hybridization-triggered cleavage agents (see, e.g., Krol et al., BioTechniques 6:958-76 (1988)) or intercalating agents. (see, e.g., Zon, Pharm. Res. 5:539-49 (1988)). To this end, the oligonucleotide may be conjugated to another molecule.

The antisense oligonucleotide may comprise at least one modified base moiety which may be selected from the group including, but not limited to, 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al., Proc. Natl. Acad. Sci. U.S.A. 93:14670 (1996) and in Eglom et al. Nature 365:566 (1993). One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual antiparallel orient, the strands run parallel to each other (Gautier et al., Nucl. Acids Res. 15:6625-41 (1987)). The oligonucleotide is a 2′-0-methylribonucleotide (Inoue et al., Nucl. Acids Res. 15:6131-48 (1987)), or a chimeric RNA-DNA analogue (Inoue et al., FEBS Lett. 215:327-30 (1987)).

Oligonucleotides of the invention may be synthesized by standard methods known in the art, e.g., by use of an automated DNA synthesizer (such as are commercially available from Biosearch Technologies, Inc. (Novato, Calif.), Applied Biosystems (Foster City, Calif.), and others). As examples, phosphorothioate oligonucleotides may be synthesized by the method of Stein et al. (Nucl. Acids Res. 16:3209 (1988)), and methylphosphonate oligonucleotides can be prepared by use of controlled pore glass polymer supports (Sarin et al., Proc. Natl. Acad. Sci. U.S.A. 85:7448-51 (1988)).

The selection of an appropriate oligonucleotide can be readily performed by one of skill in the art, based upon the present description. Given the nucleic acid encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across proteins may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific, or substantially specific, for a particular protein.

In another example, it may be desirable to design an antisense oligonucleotide that binds to and mediates the degradation of more than one message. In one example, the messages may encode related proteins such as isoforms or functionally redundant proteins. In such a case, one of skill in the art can align the nucleic acid sequences that encode these related proteins, and design an oligonucleotide that recognizes both messages.

A number of methods have been developed for delivering antisense DNA or RNA to cells, e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically to a subject. See, for example, Antisense Technology in Methods in Enzymology, Vols. 313-314, ed. by Phillips, Abelson and Simon, Academic Press, 1999.

However, in some cases, it may be difficult to achieve intracellular concentrations of the antisense molecule sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, and preferably human, cells. Such promoters can be inducible or constitutive. Such promoters include but are not limited to: the SV40 early promoter region (Bernoist and Chambon, Nature 290:304-10 (1981)), the promoter contained in the 3′ long terminal repeat of Rous sarcoma virus (Yamamoto et al., Cell 22:787-97 (1980)), the herpes thymidine kinase promoter (Wagner et al., Proc. Natl. Acad. Sci. U.S.A. 78:1441-45 (1981)), the regulatory sequences of the metallothionein gene (Brinster et al., Nature 296:39-42 (1982), and the like. Any type of plasmid, cosmid, YAC or viral vector can be used to prepare the recombinant DNA construct that can be introduced directly into the cells or tissue. Alternatively, viral vectors can be used which selectively infect the desired cells or tissue, in which case administration may be accomplished by another route (e.g., systemically).

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by any particular theory, RNAi appears to involve mRNA degradation; however, the biochemical mechanisms remain an active area of research.

As used herein, the term “dsRNA” refers to siRNA molecules, or other RNA molecules including a double stranded feature and able to be processed to siRNA in cells, such as hairpin RNA moieties.

As used herein, the phrase “mediates RNAi” refers to (indicates) the ability to distinguish, which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species, which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts, which can produce siRNAs in vivo.

“RNAi expression vector” (also referred to herein as a “dsRNA-encoding plasmid”) refers to a replicable nucleic acid constructs used to express (transcribe) RNA, which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops, which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

The sequence identity between the RNAi construct and a target sequence may be optimized by sequence comparison and alignment algorithms known in the art (see, Gribskov and Devereux, Sequence Analysis Primer, Stockton Press, 1991, and references cited therein) and by calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., using hybridization conditions such as 400 mM NaCl, 40 mM PIPES pH 6.4, 1 mM EDTA, 50° C. or 70° C. for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, e.g., Heidenreich et al., Nucleic Acids Res. 25:776-80 (1997); Wilson et al., J. Mol. Recog. 7:89-98 (1994); Chen et al., Nucleic Acids Res. 23:2661-68 (1995); Hirschbein et al., Antisense Nucleic Acid Drug Dev. 7:55-61 (1997)). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount, which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs is “small interfering RNAs” or “siRNAs.” These nucleic acids may be around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotide-long siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen et al., Proc. Natl. Acad. Sci. U.S.A., 98:9742-47 (2001); Elbashir et al., EMBO J., 20:6877-88 (2001)). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, preferably from 2 to 4 nucleotides in length. More preferably, the 3′ overhangs are 1-3 nucleotides in length. In certain embodiments, one strand has a 3′ overhang and the other strand is blunt-ended or also has an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, in some embodiments, the uses of local delivery systems and/or agents, which reduce the effects of interferon or PKR, are preferred.

In certain embodiments, the RNAi construct is in the form of a hairpin structure (named as hairpin RNA). The hairpin RNAs can be synthesized exogenously or can be formed by transcribing from RNA polymerase III or RNA polymerase II promoters in vivo. Examples of making and using such hairpin RNAs for gene silencing in mammalian cells are described in, for example, Paddison et al., Genes Dev, 16:948-58 (2002); McCaffrey et al., Nature, 418:38-39 (2002); McManus et al., RNA 8:842-50 (2002); Yu et al., Proc Natl Acad Sci U.S.A., 99:6047-52 (2002)). Preferably, such hairpin RNAs are engineered in cells or in an animal to ensure continuous and stable suppression of a desired gene. It is known in the art that siRNAs can be produced by processing a hairpin RNA in the cell. In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

International Patent Publication No. WO 01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

Exemplary RNAi constructs that specifically recognize a particular gene, or a particular family of genes can be selected using methodology outlined in detail herein with respect to the selection of antisense oligonucleotide. Similarly, methods of delivery RNAi constructs include the methods for delivery antisense oligonucleotides outlined in detail herein.

Transgenic Animals

The invention also provides transgenic non-human animals, e.g. mice, rats, rabbits, goats, sheep, dogs, cats, cows, or non-human primates, comprising, and preferably expressing, a transgene encoding a BSEP polypeptide variant. Such a transgenic animal can serve as an animal model for screening compounds having a potential toxic effect on humans.

In one embodiment, the invention provides a non-human transgenic animal whose somatic and germ cells contain a heterologous nucleic acid encoding a BSEP polypeptide variant operably-linked to a promoter, said nucleic acid comprising a segment of nucleotide at a position which corresponds to the junction between Exon I and Exon II of a human BSEP polypeptide nucleotide sequence.

As used herein, a “transgenic non-human animal” is a non-human animal in which one or more of the cells of the animal include a transgene. As used herein, the term “transgene” means a nucleic acid sequence (encoding, e.g., a BSEP polypeptide variant), which is partly or entirely heterologous, i.e., foreign, to the transgenic animal into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid.

A nucleic acid is operably linked when it is placed into a functional relationship with another nucleic acid sequence. For instance, a promoter or enhancer is operably linked to a coding sequence if it affects the transcription of said sequence.

It may be desirable to express the heterologous BSEP polypeptide variant transgene conditionally such that either the timing or the level of expression of the BSEP polypeptide variant can be regulated. Such conditional expression can be provided using prokaryotic promoter sequences, which require prokaryotic proteins to be simultaneously expressed in order to facilitate expression of the transgene. Exemplary promoters and the corresponding trans-activating prokaryotic proteins are given in U.S. Pat. No. 4,833,080.

Transgenic animals exhibiting tissue specific expression can be generated, for example, by inserting a tissue specific regulatory element, such as an enhancer, into the transgene.

Transgenic animals containing an inducible BSEP polypeptide variant transgene can be generated using inducible regulatory elements (e.g. metallothionein promoter), which are well known in the art. BSEP polypeptide variant transgene expression can then be initiated in these animals by administering to the animal a compound that induces gene expression (e.g. heavy metals). Another preferred inducible system comprises a tetracycline-inducible transcriptional activator (U.S. Pat. No. 5,654,168 issued Aug. 5, 1997 to Bujard and Gossen and U.S. Pat. No. 5,650,298 issued Jul. 22, 1997 to Bujard et al.).

In general, transgenic animal lines can be obtained by generating transgenic animals having incorporated into their genome at least one transgene, selecting at least one founder from these animals and breeding the founder or founders to establish at least one line of transgenic animals having the selected transgene incorporated into their genome. Methods of obtaining transgenic animals are well known in the art see, e.g., U.S. Pat. Nos. 4,736,866, 4,873,191, 5,602,229, 5,625,125, 5,387,742, 5,175,385, 5,175,384, 5,175,383, 6,300,540, and 5,633,425, and the references cited therein.

In one embodiment, the transgenic animals will lack the endogenous gene encoding the BSEP polypeptide. Methods for knocking-out an endogenous gene in a non-human transgenic animal are well known in the art.

Animals for obtaining eggs or other nucleated cells (e.g. embryonic stem cells) for generating transgenic animals can be obtained from standard commercial sources such as Charles River Laboratories (Wilmington, Mass.), Taconic (Germantown, N.Y.), Harlan Sprague Dawley (Indianapolis, Ind.).

Eggs can be obtained from suitable animals, e.g., by flushing from the oviduct or using techniques described in U.S. Pat. No. 5,489,742 issued Feb. 6, 1996 to Hammer and Taurog; U.S. Pat. No. 5,625,125 issued on Apr. 29, 1997 to Bennett et al.; Gordon et al., Proc. Natl. Acad. Sci. USA 77:7380-84 (1980); Gordon & Ruddle, Science 214: 1244-46 (1981); U.S. Pat. No. 4,873,191 to T. E. Wagner and P. C. Hoppe; U.S. Pat. No. 5,604,131; Armstrong et al., J. of Reproduction, 39:511 (1988) or International Patent Publication No. WO 94/00568 by Mehtali et al. Preferably, the female is subjected to hormonal conditions effective to promote superovulation prior to obtaining the eggs.

Many techniques can be used to introduce DNA into an egg or other nucleated cell, including in vitro fertilization using sperm as a carrier of exogenous DNA (“sperm-mediated gene transfer”, e.g., Lavitrano et al., Cell 57: 717-23 (1989), microinjection, gene targeting (Thompson et al., Cell 56: 313-21(1989)), electroporation (Lo, Mol. Cell. Biol. 3: 1803-14 (1983)), transfection, or retrovirus mediated gene transfer (Van der Putten et al., Proc. Natl. Acad. Sci. USA 82: 6148-52 (1985)). For a review of such techniques, see Gordon, Transgenic Animals, Intl. Rev. Cytol. 115:171-29 (1989).

Except for sperm-mediated gene transfer, eggs should be fertilized in conjunction with (before, during or after) other transgene transfer techniques. A preferred method for fertilizing eggs is by breeding the female with a fertile male. However, eggs can also be fertilized by in vitro fertilization techniques.

Fertilized, transgene containing eggs can than be transferred to pseudopregnant animals, also termed “foster mother animals”, using suitable techniques. Pseudopregnant animals can be obtained, for example, by placing 40-80 day old female animals, which are more than 8 weeks of age, in cages with infertile males, e.g., vasectomized males. The next morning females are checked for vaginal plugs. Females who have mated with vasectomized males are held aside until the time of transfer.

Recipient females can be synchronized, e.g. using the GNRH agonist (GnRH-a): des-gly10, (D-Ala6)-LH-RH Ethylamide, SigmaChemical Co., St. Louis, Mo. Alternatively, a unilateral pregnancy can be achieved by a brief surgical procedure involving the “peeling” away of the bursa membrane on the left uterine horn. Injected embryos can then be transferred to the left uterine horn via the infundibulum. Potential transgenic founders can typically be identified immediately at birth from the endogenous litter mates. For generating transgenic animals from embryonic stem cells, see e.g. Teratocarcinomas and Embryonic Stem Cells, A Practical Approach, ed. E. J. Robertson, (IRL Press 1987) or in Potter et al., Proc. Natl. Acad. Sci. USA 81:7161 (1984), the teachings of which are incorporated herein by reference.

Founders that express the gene can then bred to establish a transgenic line. Accordingly, founder animals can be bred, inbred, crossbred or outbred to produce colonies of animals of the present invention. Animals comprising multiple transgenes can be generated by crossing different founder animals (e.g. an HIV transgenic animal and a transgenic animal, which expresses human CD4), as well as by introducing multiple transgenes into an egg or embryonic cell as described above. Furthermore, embryos from A-transgenic animals can be stored as frozen embryos, which are thawed and implanted into pseudo-pregnant animals when needed (see e.g. Hirabayashi et al., Exp Anim 46: 111 (1997) and Anzai, Jikken Dobutsu 43: 247 (1994)).

The present invention provides for transgenic animals that carry the transgene in all their cells, as well as animals that carry the transgene in some, but not all cells, i.e., mosaic animals. The transgene can be integrated as a single transgene or in tandem, e.g., head to head tandems, or head to tail or tail-to-tail or as multiple copies.

The successful expression of the transgene can be detected by any of several means well known to those skilled in the art. Non-limiting examples include Northern blot, in situ hybridization of mRNA analysis, Western blot analysis, immunohistochemistry, and FACS analysis of protein expression.

The non-human transgenic animals of the present invention can serve as animal models for screening compounds having a potential toxic effect on a subject. In one embodiment, the screening assay comprises administering a test compound to a transgenic animal of the invention, and comparing a phenotypic change, such as increased toxicity or cholestasis, in the animal relative to a transgenic animal, which has not received the test compound.

In a further aspect, the invention features non-human animal cells containing a BSEP polypeptide variant transgene. For example, the animal cell (e.g. somatic cell or germ cell (i.e. egg or sperm)) can be obtained from the transgenic animal. Transgenic somatic cells or cell lines can be used, for example, in screening assays for identifying substrates and/or modulators of the BSEP polypeptide variants as described above.

Cells from the transgenic animals of the invention can be established in culture and immortalized to establish cell lines. For example, immortalized cell lines can be established from the livers of transgenic rats, as described in Bulera et al., Hepatology 25: 1192 (1997). Cell lines from other types of cells can be established according to methods known in the art.

### EXAMPLES

### Example 1

### Cloning and Sequencing of the Human BSEP Polypeptide Variants

Two oligonucleotide primers flanking the entire open reading frame (ORF) of human BSEP were chemically synthesized (Invitrogen, Carlsbad, Calif.). The forward sense oligo hBSEP-1 has the following sequence: 5′-AATT ACGCGT ACCACC ATG TCT GAC TCA GTA ATT CTT CGA AGT ATA MG AAA TTT GG-3′ (SEQ ID NO:13). The reverse oligo hBSEP-2 has the following sequence: 5′-AATT GCGGCCGC GGG TCA ACT GAT GGG GGA TCC AGT GGT GAC TAG TTT G-3′ (SEQ ID NO:14). To facilitate the cloning, Mlu I and Not I restriction enzyme sites were incorporated into the forward and reverse primer, respectively. The initiate codon in the forward primer and the stop codon in the reverse primer were bolded and underlined. The nucleic acid encoding the human BSEP polypeptide was PCR amplified using a human liver cDNA library (Clontech, Palo Alto, Calif.) as templates and Platinum Taq DNA polymerase high fidelity PCR amplification kit (Invitrogen, Carlsbad, Calif.). The PCR reaction was carried out as following: 94° C. for 2 minutes, followed by 35 cycles of 94° C. for 45 second, 54° C. for 45 second and 68° C. for 4.5 minutes, and finally extension at 68° C. for 10 minutes.

The human BSEP PCR fragments were directly cloned into pcDNA5/FRT/V5-His-TOPO expression following the protocol provided by Invitrogen. Seventeen positive clones were sequenced by the Sequencing Lab at Pfizer.

Two of the clone's encoded BSEP polypeptide insert variants and contained a segment of nucleotides at a position which corresponds to the junction between Exon I and Exon II of a human BSEP polypeptide nucleotide sequence. The sequences of these two clones are disclosed herein in SEQ ID NOs:3 and 7. SEQ ID NO:3 has extra 42 nucleotides at the junction of Exon I and II of a human BSEP polypeptide nucleotide sequence. SEQ ID NO:7 has a 102 nucleotides insertion at the junction of Exon I and II of a human BSEP polypeptide nucleotide sequence.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

Unless defined otherwise, all technical and scientific terms have the same meaning as is commonly understood by one of skill in the art to which this invention belongs.

All of the publications cited herein are hereby incorporated by reference in their entirety to describe more fully the art to which the application pertains.

All patents and publications (of any sort, including sequences cited by accession number) mentioned in the above specification are herein incorporated by reference in their entirety. Various modifications and variations of the described invention will be apparent to those skilled in the art without departing from the scope and spirit of the invention. Although the invention has been described in connection with specific preferred embodiments, it should be understood that the invention as claimed is not limited to such specific embodiments.

1. An isolated nucleic acid encoding a human BSEP polypeptide insert variant, wherein the nucleic acid comprises a segment of nucleotides at a position which corresponds to the junction between Exon I and Exon II of a human BSEP polypeptide nucleotide sequence.

2. The isolated nucleic acid of claim 1, wherein the segment of nucleotides comprises nucleotides encoding the amino acid sequence set forth in SEQ ID NO: 11.

3. The isolated nucleic acid of claim 2, wherein the segment of nucleotides comprises the nucleotide sequence set forth in SEQ ID NO:12.

4. The isolated nucleic acid of claim 1, wherein the human BSEP polypeptide insert variant comprises the amino acid sequence set forth in SEQ ID NO:4.

5. The isolated nucleic acid of claim 1, wherein the isolated nucleic acid comprises the nucleotide sequence set forth in SEQ ID NO:3.

6. The isolated nucleic acid of claim 1, wherein the human BSEP polypeptide insert variant comprises the amino acid sequence set forth in SEQ ID NO:8.

7. The isolated nucleic acid of claim 1, wherein the isolated nucleic acid comprises the nucleotide sequence set forth in SEQ ID NO:7.

8. An isolated nucleic acid encoding a BSEP polypeptide insert variant, comprising a conservative amino acid substitution with respect to the human BSEP polypeptide insert variant encoded by the isolated nucleic acid of any one of claims 4 or 6.

9. The isolated nucleic acid of claim 8, wherein the BSEP polypeptide insert variant comprises 1, 2, 3, 4, 5, 6, 7, 8, 9, or 10 conservative amino acid substitutions.

10. The isolated nucleic acid of claim 8, wherein the BSEP polypeptide insert variant comprises 11, 12, 13, 14, 15, 16, 17, 18, 19, or 20 conservative amino acid substitutions.

11. An isolated nucleic acid encoding a BSEP polypeptide insert variant, wherein the nucleic acid (i) has at least 80% sequence identity to a nucleic acid encoding a human BSEP polypeptide insert variant, and (ii) comprises a segment of nucleotides at a position which corresponds to the junction between Exon I and Exon II of a human BSEP polypeptide nucleotide sequence.

12. The isolated nucleic acid sequence of claim 11, wherein the human BSEP polypeptide variant comprises the amino acid sequence set forth in SEQ ID NO:4.

13. The isolated nucleic acid sequence of claim 11, wherein the human BSEP polypeptide variant comprises the amino acid sequence set forth in SEQ ID NO:8.

14. An isolated nucleic acid capable of hybridizing under high stringency conditions to a nucleic acid comprising a nucleic acid selected from the group consisting of:
(a) a nucleic acid encoding SEQ ID NO:6 or a fragment thereof;
(b) SEQ ID NO:5 or a fragment thereof;
(c) a nucleic acid encoding SEQ ID NO:10 or a fragment thereof;
(d) SEQ ID NO:9 or a fragment thereof;
(e) a nucleic acid encoding SEQ ID NO:11 or a fragment thereof; and
(f) SEQ ID NO:12 or a fragment thereof.

15. The nucleic acid of claim 14, wherein the nucleic acid is labeled with a detectable marker.

16. The nucleic acid of claim 15, wherein the detectable marker is a radioactive label, a calorimetric marker, a luminescent marker, an enzyme marker or a fluorescent marker.

17. A vector comprising the isolated nucleic acid of claim 1.

18. A vector comprising the isolated nucleic acid of claim 11.

19. The vector of claim 17 or 18, wherein the vector is a virus, plasmid, cosmid, λ phase or YAC.

20. A host cell comprising the nucleic acid of claim 1.

21. The host cell of claim 20, wherein the cell has been genetically engineered to comprise the nucleic acid.

22. A host cell comprising the vector of claim 17.

23. The host cell of any one of claims 20-22, wherein the cell is an eukaryotic cell.

24. The host cell of any one of claims 20-22 wherein the cell is a bacterial cell.

25. The host cell of any one of claims 20-22 wherein the cell is an insect cell.

26. The host cell of any one of claims 20-22, wherein the cell is a human cell.

27. An isolated human BSEP polypeptide insert variant encoded by a nucleic acid comprising a segment of nucleotides at a position which corresponds to the junction between Exon I and Exon II of a human BSEP polypeptide nucleotide sequence.

28. The isolated BSEP polypeptide insert variant of claim 27, wherein the segment encodes the amino acid sequence set forth in SEQ ID NO:11.

29. The isolated BSEP polypeptide insert variant of claim 27, wherein the human BSEP polypeptide insert variant comprises the amino acid sequence set forth in SEQ ID NO:4.

30. The isolated BSEP polypeptide insert variant of claim 27, wherein the human BSEP polypeptide insert variant comprises the amino acid sequence set forth in SEQ ID NO:8.

31. An isolated BSEP polypeptide insert variant comprising a conservative amino acid substitution relative to the isolated human BSEP polypeptide insert variant of any claim 29 or 30.

32. The isolated BSEP polypeptide insert variant of claim 31, comprising 1, 2, 3, 4, 5, 6, 7, 8, 9 or 10 conservative amino acid substitutions.

33. The isolated BSEP polypeptide insert variant of claim 31, comprising 11, 12, 13, 14, 15, 16, 17, 18, 19 or 20 conservative amino acid substitutions.

34. An isolated BSEP polypeptide insert variant encoded by the nucleic acid of any one of claims 11-13.

35. A method of preparing a human BSEP polypeptide insert variant comprising:
(a) culturing the host cell of claim 21 or 22 under conditions that permit expression of the BSEP polypeptide insert variant; and
(b) isolating the BSEP polypeptide insert variant, thereby preparing the BSEP polypeptide insert variant.

36. An antibody that specifically binds to the human BSEP polypeptide insert variant of claim 27 and does not bind to the human BSEP polypeptide of SEQ ID NO:2.

37. The antibody of claim 36, wherein human BSEP polypeptide insert variant is encoded by a nucleic acid comprising a segment of nucleotides encoding the amino acid sequence set forth in SEQ ID NO:11.

38. The antibody of claim 36, wherein the human BSEP polypeptide insert variant comprises the amino acid sequence set forth in SEQ ID NO:4.

39. The antibody of claim 36, wherein the human BSEP polypeptide insert variant comprises the amino acid sequence set forth in SEQ ID NO:8.

40. An antibody according to claim 38, wherein the antibody does not bind to the human BSEP polypeptide insert variant of SEQ ID NO:8.

41. An antibody according to claim 39, wherein the antibody does not bind to the human BSEP polypeptide insert variant of SEQ ID NO:4.

42. An antibody that specifically binds to the BSEP polypeptide insert variant of claim 34.

43. A method for detecting a BSEP polypeptide insert variant in a sample comprising:
(a) contacting a suitable sample with a compound that specifically binds to the BSEP polypeptide insert variant;
(b) determining whether any compound is bound to the BSEP polypeptide insert variant, wherein the presence of compound bound to the BSEP polypeptide insert variant detects the BSEP polypeptide insert variant in the sample.

44. The method of claim 43, wherein the compound is an antibody that specifically binds to the human BSEP polypeptide insert variant of SEQ ID NO:4.

45. The method of claim 43, wherein the compound is an antibody that specifically binds to the human BSEP polypeptide insert variant of SEQ ID NO:8.

46. A method for detecting a nucleic acid which encodes a BSEP polypeptide comprising:
(a) contacting a suitable sample with a compound capable of specifically binding the nucleic acid of claim 1;
(b) determining whether any compound is bound to the nucleic acid, wherein the presence of compound bound to the nucleic acid detects the nucleic acid in the sample.

47. The method of claim 46, wherein the compound is a nucleic acid capable of specifically binding to a nucleic acid encoding a BSEP polypeptide insert variant.

48. The method of claim 47, wherein the nucleic acid capable of specifically binding to a nucleic acid encoding a BSEP polypeptide insert variant is complementary to a nucleotide sequence selected from the group consisting of:
(a) a nucleic acid encoding SEQ ID NO:6 or a fragment thereof;
(b) SEQ ID NO:5 or a fragment thereof;
(c) a nucleic acid encoding SEQ ID NO:10 or a fragment thereof;
(d) SEQ ID NO:9 or a fragment thereof;
(e) a nucleic acid encoding SEQ ID NO:11 or a fragment thereof; and
(f) SEQ ID NO:12 or a fragment thereof.

49. The method of claim 43 or 44, wherein the compound is labeled with a detectable marker.

50. The method of claim 49, wherein the detectable marker is a radioactive label, a colorimetric marker, a luminescent marker, enzyme marker or a fluorescent marker.

51. A method of detecting a nucleic acid which encodes a BSEP polypeptide insert variant comprising:
(a) obtaining cDNA from mRNA obtained from a suitable sample;
(b) amplifying the cDNA corresponding to the insertion of the BSEP polypeptide insert variant or a portion of said insertion; and
(c) comparing the amplified cDNA to the DNA of a nucleic acid known to encode a BSEP polypeptide, wherein the presence of an insertion in the amplified cDNA indicates the detection of a nucleic acid encoding the BSEP polypeptide insert variant.

52. The method of claim 51, wherein step (c) is performed by comparing the sequence of the amplified cDNA to the sequence of a nucleic acid known to encode a BSEP polypeptide.

53. The method of claim 51, wherein the suitable sample is obtained from a subject, and wherein detecting the amplified cDNA indicates that the subject is expressing the BSEP polypeptide insert variant.

54. A method of determining whether a subject is expressing a BSEP polypeptide insert variant comprising:
(a) contacting a suitable sample from the subject with a compound that specifically binds to the BSEP polypeptide insert variant;
(b) determining whether any compound is bound to the BSEP polypeptide insert variant, wherein the presence of compound bound to the BSEP polypeptide insert variant indicates that the subject is expressing the BSEP polypeptide insert variant.

55. The method of claim 54, wherein the compound is an antibody that specifically binds to the human BSEP polypeptide insert variant of SEQ ID NO:4.

56. The method of claim 54, wherein the compound is an antibody that specifically binds to the human BSEP polypeptide insert variant of SEQ ID NO:8.

57. A method for determining whether a compound is a substrate or modulator of a BSEP polypeptide insert variant comprising:
(a) contacting the compound with the BSEP polypeptide insert variant; and
(b) determining whether any compound binds to the BSEP polypeptide insert variant, wherein the presence of compound bound to the BSEP polypeptide insert variant indicates that the compound is a substrate or modulator of the BSEP polypeptide insert variant.

58. A method for determining whether a compound is a substrate or modulator of a BSEP polypeptide insert variant comprising:
(a) contacting the BSEP polypeptide insert variant with a known binder and the compound;
(b) measuring the amount of binder bound to the BSEP polypeptide insert variant; and
(c) comparing the amount of binder bound to the BSEP polypeptide insert variant in step (b) with the amount of binder bound to the BSEP polypeptide insert variant in the absence of the compound, and determining whether the presence of the compound increased or decreased the amount of binder bound to the BSEP polypeptide insert variant, wherein an increase or a decrease in the amount of binder bound to the BSEP polypeptide variant indicates that the compound is a substrate or modulator of BSEP.

59. A method for determining whether a compound is a substrate or modulator of a BSEP polypeptide insert variant comprising:
(a) contacting the BSEP polypeptide insert variant with the compound and a known binder of the BSEP polypeptide insert variant;
(b) measuring the amount of compound bound to the BSEP polypeptide insert variant; and
(c) comparing the amount of binding in step (b) with the amount of binding in the absence of the known binder, and determining whether the presence of the known binder increased or decreased the binding of the compound to the BSEP polypeptide insert variant, wherein an increase or a decrease in the amount of compound bound to the BSEP polypeptide variants indicates that the compound is a substrate or modulator of BSEP.

60. The method of claim 58 or 59, wherein the known binder is selected from the group consisting of: cholyltaurine, taurocholate, taurochenodeoxycholate, taurodeoxycholate, tauroursodeoxycholate, glycohenodeoxycholate, glycocholate, lithocholate, deoxycholate, chenodeoxycholate, cholyl lysyl fluorescin AM, cholyl lysyl fluorescin diacetate, and calcein-AM.

61. The method of claim 57 or 59, wherein the compound is labeled with a detectable marker.

62. The method of claim 58, wherein the known binder is labeled with a detectable marker.

63. The method of claim 61, wherein the detectable marker is a radioactive label, a colorimetric marker, a luminescent marker, enzyme marker, a fluorescent marker or a marker that is capable of emitting electromagnetic energy.

64. The method of claim 62, wherein the detectable marker is a radioactive label, a colorimetric marker, a luminescent marker, enzyme marker, a fluorescent marker or a marker that is capable of emitting electromagnetic energy.

65. The method of claim 57, 58 or 59, wherein the binding assay is evaluated using high throughput screening.

66. A method for determining whether a compound is a substrate of a BSEP polypeptide insert variant comprising:
(a) contacting the compound with the BSEP polypeptide insert variant and ATP; and
(b) detecting the ATPase activity of the BSEP polypeptide insert variant, wherein the detection of ATPase activity indicates that the compound is a substrate of the BSEP polypeptide variant.

67. A method for determining whether a compound is a substrate or modulator of a BSEP polypeptide insert variant comprising:
(a) contacting the BSEP polypeptide insert variant with a known substrate of the BSEP polypeptide insert variant, a compound, and ATP;
(b) measuring the ATPase activity of the BSEP polypeptide insert variant; and
(c) comparing the ATPase activity of the BSEP polypeptide insert variant in step (b) with the ATPase activity of the BSEP polypeptide insert variant in the absence of the compound, wherein an increase or a decrease in the ATPase activity indicates that the compound is a substrate or modulator of the BSEP polypeptide variant.

68. The method of claim 66 or 67, wherein the assay is evaluated using high throughput screening.

69. A method for determining whether a compound is a substrate of a BSEP polypeptide insert variant comprising:
(a) contacting the compound with the BSEP polypeptide insert variant; and
(b) determining whether the compound is transported by the BSEP polypeptide insert variant, wherein the transport of the compound by the BSEP polypeptide insert variant indicates that the compound is a substrate of the BSEP polypeptide insert variant.

70. The method of claim 69, wherein the BSEP polypeptide insert variant is in a cell.

71. The method of claim 69, wherein the BSEP polypeptide insert variant is in a cell membrane vesicle.

72. The method of claim 69, wherein the BSEP polypeptide insert variant is in a cell membrane preparation.

73. The method of claim 69, wherein the compound is labeled with a detectable marker.

74. The method of claim 73, wherein the detectable marker is a radioactive label, a calorimetric marker, a luminescent marker, enzyme marker, a fluorescent marker or a marker that is capable of emitting electromagnetic energy.

75. The method of claim 69, wherein the assay is evaluated using high throughput screening.

76. A method for determining whether a compound is a substrate or modulator of a BSEP polypeptide insert variant comprising:
(a) contacting the compound with the BSEP polypeptide insert variant and a known substrate of the BSEP polypeptide insert variant;
(b) measuring the transport activity of the BSEP polypeptide insert variant with respect to the known substrate;
(c) comparing the transport activity of the BSEP polypeptide insert variant with respect to the known substrate in step (b) with the transport activity of the BSEP polypeptide with respect to the known substrate in the absence of the compound, wherein an increase or a decrease in transport of activity of the BSEP polypeptide insert variant with respect to the known substrate indicates that the compound is a substrate or modulator of the BSEP polypeptide insert variant.

77. The method of claim 76, wherein the BSEP polypeptide insert variant is in a cell.

78. The method of claim 76, wherein the BSEP polypeptide insert variant is in a cell membrane vesicle.

79. The method of claim 76, wherein the BSEP polypeptide insert variant is in a cell membrane preparation.

80. The method of claim 76, wherein the substrate is labeled with a detectable marker.

81. The method of claim 80, wherein the detectable marker is a radioactive label, a colorimetric marker, a luminescent marker, enzyme marker, a fluorescent marker or a marker that is capable of emitting electromagnetic energy.

82. The method of claim 76, wherein the substrate is selected from the group consisting of: cholyltaurine, taurocholate, taurochenodeoxycholate, taurodeoxycholate, tauroursodeoxycholate, glycohenodeoxycholate, glycocholate, lithocholate, deoxycholate, chenodeoxycholate, cholyl lysyl fluorescin AM, cholyl lysyl fluorescin diacetate, and calcein-AM.

83. The method of claim 76, wherein the assay is evaluated using high throughput screening.

84. A method of inhibiting the expression of a human BSEP polypeptide insert variant in a cell comprising contacting the cell with a compound capable of inhibiting the expression of the human BSEP polypeptide insert variant.

85. The method of claim 84, wherein the compound is an antisense oligonucleotide capable of specifically hybridizing to the BSEP polypeptide insert variant.

86. The method of claim 85, wherein the antisense oligonucleotide is capable of specifically hybridizing to a nucleic acid sequence selected from the group consisting of:
(a) a nucleic acid encoding SEQ ID NO:6 or a fragment thereof;
(b) SEQ ID NO:5 or a fragment thereof;
(c) a nucleic acid encoding SEQ ID NO:10 or a fragment thereof;
(d) SEQ ID NO:9 or a fragment thereof;
(e) a nucleic acid encoding SEQ ID NO:11 or a fragment thereof; and
(f) SEQ ID NO:12 or a fragment thereof.

87. The method of claim 84, wherein the compound is an RNAi construct capable of specifically hybridizing to the BSEP polypeptide insert variant.

88. The method of claim 87, wherein the RNAi construct is capable of specifically hybridizing to a nucleic acid sequence selected from the group consisting of:
(a) a nucleic acid sequence SEQ ID NO:6 or a fragment thereof;
(b) SEQ ID NO:5 or a fragment thereof;
(c) a nucleic acid encoding SEQ ID NO:10 or a fragment thereof;
(d) SEQ ID NO:9 or a fragment thereof;
(e) a nucleic acid encoding SEQ ID NO:11 or a fragment thereof; and
(f) SEQ ID NO:12 or a fragment thereof.

89. A non-human transgenic animal whose somatic and germ cells contain a heterologous nucleic acid encoding a BSEP polypeptide variant operably-linked to a promoter, said nucleic acid comprising a segment of nucleotide at a position which corresponds to the junction between Exon I and Exon II of a human BSEP polypeptide nucleotide sequence.


SEQUENCE LISTING (14 sequences)

SEQ ID NO: 1 - DNA, 3966 bp, Homo sapiens
  CDS (1)..(3966)
atgtctgactcagtaattcttcgaagtataaagaaatttggagaggagaatgatggttttgagtcagataaatcatataataatgataagaaatcaaggttacaagatgagaagaaaggtgatggcgttagagttggcttctttcaattgtttcggttttcttcatcaactgacatttggctgatgtttgtgggaagtttgtgtgcatttctccatggaatagcccagccaggcgtgctactcatttttggcacaatgacagatgtttttattgactacgacgttgagttacaagaactccagattccaggaaaagcatgtgtgaataacaccattgtatggactaacagttccctcaaccagaacatgacaaatggaacacgttgtgggttgctgaacatcgagagcgaaatgatcaaatttgccagttactatgctggaattgctgtcgcagtacttatcacaggatatattcaaatatgcttttgggtcattgccgcagctcgtcagatacagaaaatgagaaaattttactttaggagaataatgagaatggaaatagggtggtttgactgcaattcagtgggggagctgaatacaagattctctgatgatattaataaaatcaatgatgccatagctgaccaaatggcccttttcattcagcgcatgacctcgaccatctgtggtttcctgttgggatttttcaggggttggaaactgaccttggttattatttctgtcagccctctcattgggattggagcagccaccattggtctgagtgtgtccaagtttacggactatgagctgaaggcctatgccaaagcaggggtggtggctgatgaagtcatttcatcaatgagaacagtggctgcttttggtggtgagaaaagagaggttgaaaggtatgagaaaaatcttgtgttcgcccagcgttggggaattagaaaaggaatagtgatgggattctttactggattcgtgtggtgtctcatctttttgtgttatgcactggccttctggtacggctccacacttgtcctggatgaaggagaatatacaccaggaacccttgtccagattttcctcagtgtcatagtaggagctttaaatcttggcaatgcctctccttgtttggaagcctttgcaactggacgtgcagcagccaccagcatttttgagacaatagacaggaaacccatcattgactgcatgtcagaagatggttacaagttggatcgaatcaagggtgaaattgaattccataatgtgaccttccattatccttccagaccagaggtgaagattctaaatgacctcaacatggtcattaaaccaggggaaatgacagctctggtaggacccagtggagctggaaaaagtacagcactgcaactcattcagcgattctatgacccctgtgaaggaatggtgaccgtggatggccatgacattcgctctcttaacattcagtggcttagagatcagattgggatagtggagcaagagccagttctgttctctaccaccattgcagaaaatattcgctatggcagagaagatgcaacaatggaagacatagtccaagctgccaaggaggccaatgcctacaacttcatcatggacctgccacagcaatttgacacccttgttggagaaggaggaggccagatgagtggtggccagaaacaaagggtagctatcgccagagccctcatccgaaatcccaagattctgcttttggacatggccacctcagctctggacaatgagagtgaagccatggtgcaagaagtgctgagtaagattcagcatgggcacacaatcatttcagttgctcatcgcttgtctacggtcagagctgcagataccatcattggttttgaacatggcactgcagtggaaagagggacccatgaagaattactggaaaggaaaggtgtttacttcactctagtgactttgcaaagccagggaaatcaagctcttaatgaagaggacataaaggatgcaactgaagatgacatgcttgcgaggacctttagcagagggagctaccaggatagtttaagggcttccatccggcaacgctccaagtctcagctttcttacctggtgcacgaacctccattagctgttgtagatcataagtctacctatgaagaagatagaaaggacaaggacattcctgtgcaggaagaagttgaacctgccccagttaggaggattctgaaattcagtgctccagaatggccctacatgctggtagggtctgtgggtgcagctgtgaacgggacagtcacacccttgtatgcctttttattcagccagattcttgggactttttcaattcctgataaagaggaacaaaggtcacagatcaatggtgtgtgcctactttttgtagcaatgggctgtgtatctcttttcacccaatttctacagggatatgcctttgctaaatctggggagctcctaacaaaaaggctacgtaaatttggtttcagggcaatgctggggcaagatattgcctggtttgatgacctcagaaatagccctggagcattgacaacaagacttgctacagatgcttcccaagttcaaggggctgccggctctcagatcgggatgatagtcaattccttcactaacgtcactgtggccatgatcattgccttctcctttagctggaagctgagcctggtcatcttgtgcttcttccccttcttggctttatcaggagccacacagaccaggatgttgacaggatttgcctctcgagataagcaggccctggagatggtgggacagattacaaatgaagccctcagtaacatccgcactgttgctggaattggaaaggagaggcggttcattgaagcacttgagactgagctggagaagcccttcaagacagccattcagaaagccaatatttacggattctgctttgcctttgcccagtgcatcatgtttattgcgaattctgcttcctacagatatggaggttacttaatctccaatgaggggctccatttcagctatgtgttcagggtgatctctgcagttgtactgagtgcaacagctcttggaagagccttctcttacaccccaagttatgcaaaagctaaaatatcagctgcacgcttttttcaactgctggaccgacaacccccaatcagtgtatacaatactgcaggtgaaaaatgggacaacttccaggggaagattgattttgttgattgtaaatttacatatccttctcgacctgactcgcaagttctgaatggtctctcagtgtcgattagtccagggcagacactggcgtttgttgggagcagtggatgtggcaaaagcactagcattcagctgttggaacgtttctatgatcctgatcaagggaaggtgatgatagatggtcatgacagcaaaaaagtaaatgtccagttcctccgctcaaacattggaattgtttcccaggaaccagtgttgtttgcctgtagcataatggacaatatcaagtatggagacaacaccaaagaaattcccatggaaagagtcatagcagctgcaaaacaggctcagctgcatgattttgtcatgtcactcccagagaaatatgaaactaacgttgggtcccaggggtctcaactctctagaggggagaaacaacgcattgctattgctcgggccattgtacgagatcctaaaatcttgctactagatgaagccacttctgccttagacacagaaagtgaaaagacggtgcaggttgctctagacaaagccagagagggtcggacctgcattgtcattgcccatcgcttgtccaccatccagaacgcggatatcattgctgtcatggcacagggggtggtgattgaaaaggggacccatgaagaactgatggcccaaaaaggagcctactacaaactagtcaccactggatcccccatcagttga

SEQ ID NO: 2 - protein, 1321 aa, Homo sapiens
MSDSVILRSIKKFGEENDGFESDKSYNNDKKSRLQDEKKGDGVRVGFFQLFRFSSSTDIWLMFVGSLCAFLHGIAQPGVLLIFGTMTDVFIDYDVELQELQIPGKACVNNTIVWTNSSLNQNMTNGTRCGLLNIESEMIKFASYYAGIAVAVLITGYIQICFWVIAAARQIQKMRKFYFRRIMRMEIGWFDCNSVGELNTRFSDDINKINDAIADQMALFIQRMTSTICGFLLGFFRGWKLTLVIISVSPLIGIGAATIGLSVSKFTDYELKAYAKAGVVADEVISSMRTVAAFGGEKREVERYEKNLVFAQRWGIRKGIVMGFFTGFVWCLIFLCYALAFWYGSTLVLDEGEYTPGTLVQIFLSVIVGALNLGNASPCLEAFATGRAAATSIFETIDRKPIIDCMSEDGYKLDRIKGEIEFHNVTFHYPSRPEVKILNDLNMVIKPGEMTALVGPSGAGKSTALQLIQRFYDPCEGMVTVDGHDIRSLNIQWLRDQIGIVEQEPVLFSTTIAENIRYGREDATMEDIVQAAKEANAYNFIMDLPQQFDTLVGEGGGQMSGGQKQRVAIARALIRNPKILLLDMATSALDNESEAMVQEVLSKIQHGHTIISVAHRLSTVRAADTIIGFEHGTAVERGTHEELLERKGVYFTLVTLQSQGNQALNEEDIKDATEDDMLARTFSRGSYQDSLRASIRQRSKSQLSYLVHEPPLAVVDHKSTYEEDRKDKDIPVQEEVEPAPVRRILKFSAPEWPYMLVGSVGAAVNGTVTPLYAFLFSQILGTFSIPDKEEQRSQINGVCLLFVAMGCVSLFTQFLQGYAFAKSGELLTKRLRKFGFRAMLGQDIAWFDDLRNSPGALTTRLATDASQVQGAAGSQIGMIVNSFTNVTVAMIIAFSFSWKLSLVILCFFPFLALSGATQTRMLTGFASRDKQALEMVGQITNEALSNIRTVAGIGKERRFIEALETELEKPFKTAIQKANIYGFCFAFAQCIMFIANSASYRYGGYLISNEGLHFSYVFRVISAVVLSATALGRAFSYTPSYAKAKISAARFFQLLDRQPPISVYNTAGEKWDNFQGKIDFVDCKFTYPSRPDSQVLNGLSVSISPGQTLAFVGSSGCGKSTSIQLLERFYDPDQGKVMIDGHDSKKVNVQFLRSNIGIVSQEPVLFACSIMDNIKYGDNTKEIPMERVIAAAKQAQLHDFVMSLPEKYETNVGSQGSQLSRGEKQRIAIARAIVRDPKILLLDEATSALDTESEKTVQVALDKAREGRTCIVIAHRLSTIQNADIIAVMAQGVVIEKGTHEELMAQKGAYYKLVTTGSPIS

SEQ ID NO: 3 - DNA, 4008 bp, Homo sapiens
  CDS (1)..(4008)
atgtctgactcagtaattcttcgaagtataaagaaatttggagaggagaatgatggttttgagtcagataaatcatgtgagtggcttttttccctcactgcatcttgtacaaggagagataataatgataagaaatcaaggttacaagatgagaagaaaggtgatggcgttagagttggcttctttcaattgtttcggttttcttcatcaactgacatttggctgatgtttgtgggaagtttgtgtgcatttctccatggaatagcccagccaggcgtgctactcatttttggcacaatgacagatgtttttattgactacgacgttgagttacaagaactccagattccaggaaaagcatgtgtgaataacaccattgtatggactaacagttccctcaaccagaacatgacaaatggaacacgttgtgggttgctgaacatcgagagcgaaatgatcaaatttgccagttactatgctggaattgctgtcgcagtacttatcacaggatatattcaaatatgcttttgggtcattgccgcagctcgtcagatacagaaaatgagaaaattttactttaggagaataatgagaatggaaatagggtggtttgactgcaattcagtgggggagctgaatacaagattctctgatgatattaataaaatcaatgatgccatagctgaccaaatggcccttttcattcagcgcatgacctcgaccatctgtggtttcctgttgggatttttcaggggttggaaactgaccttggttattatttctgtcagccctctcattgggattggagcagccaccattggtctgagtgtgtccaagtttacggactatgagctgaaggcctatgccaaagcaggggtggtggctgatgaagtcatttcatcaatgagaacagtggctgcttttggtggtgagaaaagagaggttgaaaggtatgagaaaaatcttgtgttcgcccagcgttggggaattagaaaaggaatagtgatgggattctttactggattcgtgtggtgtctcatctttttgtgttatgcactggccttctggtacggctccacacttgtcctggatgaaggagaatatacaccaggaacccttgtccagattttcctcagtgtcatagtaggagctttaaatcttggcaatgcctctccttgtttggaagcctttgcaactggacgtgcagcagccaccagcatttttgagacaatagacaggaaacccatcattgactgcatgtcagaagatggttacaagttggatcgaatcaagggtgaaattgaattccataatgtgaccttccattatccttccagaccagaggtgaagattctaaatgacctcaacatggtcattaaaccaggggaaatgacagctctggtaggacccagtggagctggaaaaagtacagcactgcaactcattcagcgattctatgacccctgtgaaggaatggtgaccgtggatggccatgacattcgctctcttaacattcagtggcttagagatcagattgggatagtggagcaagagccagttctgttctctaccaccattgcagaaaatattcgctatggcagagaagatgcaacaatggaagacatagtccaagctgccaaggaggccaatgcctacaacttcatcatggacctgccacagcaatttgacacccttgttggagaaggaggaggccagatgagtggtggccagaaacaaagggtagctatcgccagagccctcatccgaaatcccaagattctgcttttggacatggccacctcagctctggacaatgagagtgaagccatggtgcaagaagtgctgagtaagattcagcatgggcacacaatcatttcagttgctcatcgcttgtctacggtcagagctgcagataccatcattggttttgaacatggcactgcagtggaaagagggacccatgaagaattactggaaaggaaaggtgtttacttcactctagtgactttgcaaagccagggaaatcaagctcttaatgaagaggacataaaggatgcaactgaagatgacatgcttgcgaggacctttagcagagggagctaccaggatagtttaagggcttccatccggcaacgctccaagtctcagctttcttacctggtgcacgaacctccattagctgttgtagatcataagtctacctatgaagaagatagaaaggacaaggacattcctgtgcaggaagaagttgaacctgccccagttaggaggattctgaaattcagtgctccagaatggccctacatgctggtagggtctgtgggtgcagctgtgaacgggacagtcacacccttgtatgcctttttattcagccagattcttgggactttttcaattcctgataaagaggaacaaaggtcacagatcaatggtgtgtgcctactttttgtagcaatgggctgtgtatctcttttcacccaatttctacagggatatgcctttgctaaatctggggagctcctaacaaaaaggctacgtaaatttggtttcagggcaatgctggggcaagatattgcctggtttgatgacctcagaaatagccctggagcattgacaacaagacttgctacagatgcttcccaagttcaaggggctgccggctctcagatcgggatgatagtcaattccttcactaacgtcactgtggccatgatcattgccttctcctttagctggaagctgagcctggtcatcttgtgcttcttccccttcttggctttatcaggagccacacagaccaggatgttgacaggatttgcctctcgagataagcaggccctggagatggtgggacagattacaaatgaagccctcagtaacatccgcactgttgctggaattggaaaggagaggcggttcattgaagcacttgagactgagctggagaagcccttcaagacagccattcagaaagccaatatttacggattctgctttgcctttgcccagtgcatcatgtttattgcgaattctgcttcctacagatatggaggttacttaatctccaatgaggggctccatttcagctatgtgttcagggtgatctctgcagttgtactgagtgcaacagctcttggaagagccttctcttacaccccaagttatgcaaaagctaaaatatcagctgcacgcttttttcaactgctggaccgacaacccccaatcagtgtatacaatactgcaggtgaaaaatgggacaacttccaggggaagattgattttgttgattgtaaatttacatatccttctcgacctgactcgcaagttctgaatggtctctcagtgtcgattagtccagggcagacactggcgtttgttgggagcagtggatgtggcaaaagcactagcattcagctgttggaacgtttctatgatcctgatcaagggaaggtgatgatagatggtcatgacagcaaaaaagtaaatgtccagttcctccgctcaaacattggaattgtttcccaggaaccagtgttgtttgcctgtagcataatggacaatatcaagtatggagacaacaccaaagaaattcccatggaaagagtcatagcagctgcaaaacaggctcagctgcatgattttgtcatgtcactcccagagaaatatgaaactaacgttgggtcccaggggtctcaactctctagaggggagaaacaacgcattgctattgctcgggccattgtacgagatcctaaaatcttgctactagatgaagccacttctgccttagacacagaaagtgaaaagacggtgcaggttgctctagacaaagccagagagggtcggacctgcattgtcattgcccatcgcttgtccaccatccagaacgcggatatcattgctgtcatggcacagggggtggtgattgaaaaggggacccatgaagaactgatggcccaaaaaggagcctactacaaactagtcaccactggatcccccatcagttga

SEQ ID NO: 4 - protein, 1335 aa, Homo sapiens
MSDSVILRSIKKFGEENDGFESDKSCEWLFSLTASCTRRDNNDKKSRLQDEKKGDGVRVGFFQLFRFSSSTDIWLMFVGSLCAFLHGIAQPGVLLIFGTMTDVFIDYDVELQELQIPGKACVNNTIVWTNSSLNQNMTNGTRCGLLNIESEMIKFASYYAGIAVAVLITGYIQICFWVIAAARQIQKMRKFYFRRIMRMEIGWFDCNSVGELNTRFSDDINKINDAIADQMALFIQRMTSTICGFLLGFFRGWKLTLVIISVSPLIGIGAATIGLSVSKFTDYELKAYAKAGVVADEVISSMRTVAAFGGEKREVERYEKNLVFAQRWGIRKGIVMGFFTGFVWCLIFLCYALAFWYGSTLVLDEGEYTPGTLVQIFLSVIVGALNLGNASPCLEAFATGRAAATSIFETIDRKPIIDCMSEDGYKLDRIKGEIEFHNVTFHYPSRPEVKILNDLNMVIKPGEMTALVGPSGAGKSTALQLIQRFYDPCEGMVTVDGHDIRSLNIQWLRDQIGIVEQEPVLFSTTIAENIRYGREDATMEDIVQAAKEANAYNFIMDLPQQFDTLVGEGGGQMSGGQKQRVAIARALIRNPKILLLDMATSALDNESEAMVQEVLSKIQHGHTIISVAHRLSTVRAADTIIGFEHGTAVERGTHEELLERKGVYFTLVTLQSQGNQALNEEDIKDATEDDMLARTFSRGSYQDSLRASIRQRSKSQLSYLVHEPPLAVVDHKSTYEEDRKDKDIPVQEEVEPAPVRRILKFSAPEWPYMLVGSVGAAVNGTVTPLYAFLFSQILGTFSIPDKEEQRSQINGVCLLFVAMGCVSLFTQFLQGYAFAKSGELLTKRLRKFGFRAMLGQDIAWFDDLRNSPGALTTRLATDASQVQGAAGSQIGMIVNSFTNVTVAMIIAFSFSWKLSLVILCFFPFLALSGATQTRMLTGFASRDKQALEMVGQITNEALSNIRTVAGIGKERRFIEALETELEKPFKTAIQKANIYGFCFAFAQCIMFIANSASYRYGGYLISNEGLHFSYVFRVISAVVLSATALGRAFSYTPSYAKAKISAARFFQLLDRQPPISVYNTAGEKWDNFQGKIDFVDCKFTYPSRPDSQVLNGLSVSISPGQTLAFVGSSGCGKSTSIQLLERFYDPDQGKVMIDGHDSKKVNVQFLRSNIGIVSQEPVLFACSIMDNIKYGDNTKEIPMERVIAAAKQAQLHDFVMSLPEKYETNVGSQGSQLSRGEKQRIAIARAIVRDPKILLLDEATSALDTESEKTVQVALDKAREGRTCIVIAHRLSTIQNADIIAVMAQGVVIEKGTHEELMAQKGAYYKLVTTGSPIS

SEQ ID NO: 5 - DNA, 42 bp, Homo sapiens
gtgagtggcttttttccctcactgcatcttgtacaaggagag

SEQ ID NO: 6 - protein, 15 aa, Homo sapiens
CEWLFSLTASCTRRD

SEQ ID NO: 7 - DNA, 4068 bp, Homo sapiens
  CDS (1)..(4068)
atgtctgactcagtaattcttcgaagtataaagaaatttggagaggagaatgatggttttgagtcagataaatcatgtgagtggcttttttccctcactgcatcttgtacaaggagaggtgagaacaaaagtaggacaagctggtcaagtttcaaggagcagaaaaaaatcagcaacaataataatgataagaaatcaaggttacaagatgagaagaaaggtgatggcgttagagttggcttctttcaattgtttcggttttcttcatcaactgacatttggctgatgtttgtgggaagtttgtgtgcatttctccatggaatagcccagccaggcgtgctactcatttttggcacaatgacagatgtttttattgactacgacgttgagttacaagaactccagattccaggaaaagcatgtgtgaataacaccattgtatggactaacagttccctcaaccagaacatgacaaatggaacacgttgtgggttgctgaacatcgagagcgaaatgatcaaatttgccagttactatgctggaattgctgtcgcagtacttatcacaggatatattcaaatatgcttttgggtcattgccgcagctcgtcagatacagaaaatgagaaaattttactttaggagaataatgagaatggaaatagggtggtttgactgcaattcagtgggggagctgaatacaagattctctgatgatattaataaaatcaatgatgccatagctgaccaaatggcccttttcattcagcgcatgacctcgaccatctgtggtttcctgttgggatttttcaggggttggaaactgaccttggttattatttctgtcagccctctcattgggattggagcagccaccattggtctgagtgtgtccaagtttacggactatgagctgaaggcctatgccaaagcaggggtggtggctgatgaagtcatttcatcaatgagaacagtggctgcttttggtggtgagaaaagagaggttgaaaggtatgagaaaaatcttgtgttcgcccagcgttggggaattagaaaaggaatagtgatgggattctttactggattcgtgtggtgtctcatctttttgtgttatgcactggccttctggtacggctccacacttgtcctggatgaaggagaatatacaccaggaacccttgtccagattttcctcagtgtcatagtaggagctttaaatcttggcaatgcctctccttgtttggaagcctttgcaactggacgtgcagcagccaccagcatttttgagacaatagacaggaaacccatcattgactgcatgtcagaagatggttacaagttggatcgaatcaagggtgaaattgaattccataatgtgaccttccattatccttccagaccagaggtgaagattctaaatgacctcaacatggtcattaaaccaggggaaatgacagctctggtaggacccagtggagctggaaaaagtacagcactgcaactcattcagcgattctatgacccctgtgaaggaatggtgaccgtggatggccatgacattcgctctcttaacattcagtggcttagagatcagattgggatagtggagcaagagccagttctgttctctaccaccattgcagaaaatattcgctatggcagagaagatgcaacaatggaagacatagtccaagctgccaaggaggccaatgcctacaacttcatcatggacctgccacagcaatttgacacccttgttggagaaggaggaggccagatgagtggtggccagaaacaaagggtagctatcgccagagccctcatccgaaatcccaagattctgcttttggacatggccacctcagctctggacaatgagagtgaagccatggtgcaagaagtgctgagtaagattcagcatgggcacacaatcatttcagttgctcatcgcttgtctacggtcagagctgcagataccatcattggttttgaacatggcactgcagtggaaagagggacccatgaagaattactggaaaggaaaggtgtttacttcactctagtgactttgcaaagccagggaaatcaagctcttaatgaagaggacataaaggatgcaactgaagatgacatgcttgcgaggacctttagcagagggagctaccaggatagtttaagggcttccatccggcaacgctccaagtctcagctttcttacctggtgcacgaacctccattagctgttgtagatcataagtctacctatgaagaagatagaaaggacaaggacattcctgtgcaggaagaagttgaacctgccccagttaggaggattctgaaattcagtgctccagaatggccctacatgctggtagggtctgtgggtgcagctgtgaacgggacagtcacacccttgtatgcctttttattcagccagattcttgggactttttcaattcctgataaagaggaacaaaggtcacagatcaatggtgtgtgcctactttttgtagcaatgggctgtgtatctcttttcacccaatttctacagggatatgcctttgctaaatctggggagctcctaacaaaaaggctacgtaaatttggtttcagggcaatgctggggcaagatattgcctggtttgatgacctcagaaatagccctggagcattgacaacaagacttgctacagatgcttcccaagttcaaggggctgccggctctcagatcgggatgatagtcaattccttcactaacgtcactgtggccatgatcattgccttctcctttagctggaagctgagcctggtcatcttgtgcttcttccccttcttggctttatcaggagccacacagaccaggatgttgacaggatttgcctctcgagataagcaggccctggagatggtgggacagattacaaatgaagccctcagtaacatccgcactgttgctggaattggaaaggagaggcggttcattgaagcacttgagactgagctggagaagcccttcaagacagccattcagaaagccaatatttacggattctgctttgcctttgcccagtgcatcatgtttattgcgaattctgcttcctacagatatggaggttacttaatctccaatgaggggctccatttcagctatgtgttcagggtgatctctgcagttgtactgagtgcaacagctcttggaagagccttctcttacaccccaagttatgcaaaagctaaaatatcagctgcacgcttttttcaactgctggaccgacaacccccaatcagtgtatacaatactgcaggtgaaaaatgggacaacttccaggggaagattgattttgttgattgtaaatttacatatccttctcgacctgactcgcaagttctgaatggtctctcagtgtcgattagtccagggcagacactggcgtttgttgggagcagtggatgtggcaaaagcactagcattcagctgttggaacgtttctatgatcctgatcaagggaaggtgatgatagatggtcatgacagcaaaaaagtaaatgtccagttcctccgctcaaacattggaattgtttcccaggaaccagtgttgtttgcctgtagcataatggacaatatcaagtatggagacaacaccaaagaaattcccatggaaagagtcatagcagctgcaaaacaggctcagctgcatgattttgtcatgtcactcccagagaaatatgaaactaacgttgggtcccaggggtctcaactctctagaggggagaaacaacgcattgctattgctcgggccattgtacgagatcctaaaatcttgctactagatgaagccacttctgccttagacacagaaagtgaaaagacggtgcaggttgctctagacaaagccagagagggtcggacctgcattgtcattgcccatcgcttgtccaccatccagaacgcggatatcattgctgtcatggcacagggggtggtgattgaaaaggggacccatgaagaactgatggcccaaaaaggagcctactacaaactagtcaccactggatcccccatcagttga

SEQ ID NO: 8 - protein, 1355 aa, Homo sapiens
MSDSVILRSIKKFGEENDGFESDKSCEWLFSLTASCTRRGENKSRTSWSSFKEQKKISNNNNDKKSRLQDEKKGDGVRVGFFQLFRFSSSTDIWLMFVGSLCAFLHGIAQPGVLLIFGTMTDVFIDYDVELQELQIPGKACVNNTIVWTNSSLNQNMTNGTRCGLLNIESEMIKFASYYAGIAVAVLITGYIQICFWVIAAARQIQKMRKFYFRRIMRMEIGWFDCNSVGELNTRFSDDINKINDAIADQMALFIQRMTSTICGFLLGFFRGWKLTLVIISVSPLIGIGAATIGLSVSKFTDYELKAYAKAGVVADEVISSMRTVAAFGGEKREVERYEKNLVFAQRWGIRKGIVMGFFTGFVWCLIFLCYALAFWYGSTLVLDEGEYTPGTLVQIFLSVIVGALNLGNASPCLEAFATGRAAATSIFETIDRKPIIDCMSEDGYKLDRIKGEIEFHNVTFHYPSRPEVKILNDLNMVIKPGEMTALVGPSGAGKSTALQLIQRFYDPCEGMVTVDGHDIRSLNIQWLRDQIGIVEQEPVLFSTTIAENIRYGREDATMEDIVQAAKEANAYNFIMDLPQQFDTLVGEGGGQMSGGQKQRVAIARALIRNPKILLLDMATSALDNESEAMVQEVLSKIQHGHTIISVAHRLSTVRAADTIIGFEHGTAVERGTHEELLERKGVYFTLVTLQSQGNQALNEEDIKDATEDDMLARTFSRGSYQDSLRASIRQRSKSQLSYLVHEPPLAVVDHKSTYEEDRKDKDIPVQEEVEPAPVRRILKFSAPEWPYMLVGSVGAAVNGTVTPLYAFLFSQILGTFSIPDKEEQRSQINGVCLLFVAMGCVSLFTQFLQGYAFAKSGELLTKRLRKFGFRAMLGQDIAWFDDLRNSPGALTTRLATDASQVQGAAGSQIGMIVNSFTNVTVAMIIAFSFSWKLSLVILCFFPFLALSGATQTRMLTGFASRDKQALEMVGQITNEALSNIRTVAGIGKERRFIEALETELEKPFKTAIQKANIYGFCFAFAQCIMFIANSASYRYGGYLISNEGLHFSYVFRVISAVVLSATALGRAFSYTPSYAKAKISAARFFQLLDRQPPISVYNTAGEKWDNFQGKIDFVDCKFTYPSRPDSQVLNGLSVSISPGQTLAFVGSSGCGKSTSIQLLERFYDPDQGKVMIDGHDSKKVNVQFLRSNIGIVSQEPVLFACSIMDNIKYGDNTKEIPMERVIAAAKQAQLHDFVMSLPEKYETNVGSQGSQLSRGEKQRIAIARAIVRDPKILLLDEATSALDTESEKTVQVALDKAREGRTCIVIAHRLSTIQNADIIAVMAQGVVIEKGTHEELMAQKGAYYKLVTTGSPIS

SEQ ID NO: 9 - DNA, 102 bp, Homo sapiens
gtgagtggcttttttccctcactgcatcttgtacaaggagaggtgagaacaaaagtaggacaagctggtcaagtttcaaggagcagaaaaaaatcagcaaca

SEQ ID NO: 10 - protein, 35 aa, Homo sapiens
CEWLFSLTASCTRRGENKSRTSWSSFKEQKKISNN

SEQ ID NO: 11 - protein, 13 aa, Homo sapiens
EWLFSLTASCTRR

SEQ ID NO: 12 - DNA, 42 bp, Homo sapiens; identical to SEQ ID NO: 5

SEQ ID NO: 13 - DNA, 57 bp, Artificial Sequence
  forward sense oligo hBSEP-1
aattacgcgtaccaccatgtctgactcagtaattcttcgaagtataaagaaatttgg

SEQ ID NO: 14 - DNA, 49 bp, Artificial Sequence
  reverse oligo hBSEP-2
aattgcggccgcgggtcaactgatgggggatccagtggtgactagtttg